(19) United States
(12) Patent Application Publication
(10) Pub. No.: US 2016/0230142 A1
(43) Pub. Date: Aug. 11, 2016

(54) CORNEAL STROMAL KERATOCYTE CULTURE

(71) Applicant: SINGAPORE HEALTH SERVIES PTE LTD., Singapore (SG)

(72) Inventors: Hin-Fai Gary YAM, Singapore (SG); Jodhbir MEHTA, Singapore (SG)

(73) Assignees: Singapore Health Services Pte Ltd, Singapore (SG); Singapore Health Services Pte Ltd, Singapore (SG)

(21) Appl. No.: 15/022,556

(22) PCT Filed: Sep. 17, 2014
(86) PCT No.: PCT/SG2014/000445

§ 371 Date: Mar. 16, 2016

(30) Foreign Application Priority Data
Sep. 17, 2013 (SG) 201307106-3

(51) Int. Cl. C12N 5/079

(52) U.S. Cl. CPC C12N 5/0621; C12N 2500/84; C12N 2501/727; C12N 2501/105; C12N 2533/54

(57) ABSTRACT

The present invention relates to corneal stromal keratocytes (CSKs) culture. In particular the present invention relates to a method for culturing corneal stromal keratocytes ex vivo. The method utilises a dual culture medium protocol. In the presence of serum (in particular low serum concentration) in a first culture medium A, the “activated keratocytes” are expandable while specific CSK gene expression is maintained in serum-free ERI condition in a second culture medium B. The invention also relates to culture medium A and B which is supplemented with liquid amnion extract or other additional supplements. This protocol also applied to CSK from other species. Additionally, the present invention provides method for CSK cultivation to provide sufficient quantity of genuine CSKs for corneal tissue engineering without a risk of fibroblastic changes.

### FIELD OF THE INVENTION

The present invention relates to the field of cell culture, tissue culture and tissue engineering. In particular, the invention relates to methods and systems for culturing cells ex vivo or in vitro through the use of various conditions and agents, to control the growth and/or development of the cells; maintain cellular morphology and/or phenotype; and/or prevent changes (e.g. physical changes) to the cells.

### BACKGROUND OF THE INVENTION

The cornea is a transparent, avascular structure in the anterior part of the eye. Besides acting as a protective barrier, it provides 70% of refractive power to converge incoming light to the lens and retina. It contains 3 major layers: an outermost non-keratinized stratified epithelium, a middle collagen-rich stroma and an inner single cell-layered endothelium. The stroma spans about 90% of corneal thickness and consists of transparent extracellular matrix deposited by the resident corneal stromal keratocytes (CSKs). CSKs are neural crest-derived mesenchymal cells and mitotically quiescent. CSKs are sparsely located in the stroma matrix and exhibit a flattened, dendritic morphology with extensive cellular contacts with neighboring CSKs, through gap junctions, thus forming a 3D network. During corneal development, CSKs are biosynthetically active, producing fibrillar collagens and keratan sulfate-proteoglycans that assemble into a highly organized extracellular matrix with uniform collagen fibrils and interfibrillar spacing, which is required for a transparent cornea. CSKs express specific proteins, including stromal crystallins (aldehyde dehydrogenases and transketolase) and keratan sulfate-proteoglycans (lumican, keratocan and mimecan).

Corneal or stromal damages, such as by physical injury or infection, cause CSKs in the wound site to undergo apoptosis. Peripheral keratocytes become activated with a transient stage of “activated keratocytes”, typified by a loss of stromal crystallins. They further transit into repair fibroblasts, which proliferate and migrate to the injury site. These repair fibroblasts lose all keratocyte features. Repair fibroblast cells are spindle in shape, with long, spreading cellular processes and actively produce new stromal matrix proteins, including collagens and proteoglycans as well as matrix metalloproteinase-1, 3 and 9, fibronectin and α5-integrin, which are not detectable in normal stroma. Some fibroblasts eventually transform into myofibroblasts under the synergistic interaction of serum factors (such as transforming growth factor β (TGFβ) and platelet-derived growth factor). Myofibroblasts are rich in α-smooth muscle actin (αSMA). Through the smooth muscle-like contractile mechanism, the interwoven network of cells and extracellular matrix (ECM) contract and form scars accompanied by myofibroblast apoptosis. In human, corneal scars can remain for decades. There have been extensive reports to elucidate the nature of stromal fibroblasts and their regulatory mechanisms in the wound healing process of cornea. However, corneal tissue engineering requires the use of genuine CSKs, which should be capable to propagate ex vivo without any loss of keratocyte properties. In serum-free culture supplemented with insulin, selenium and transferrin, the stromal cells can maintain keratocyte phenotype and in the presence of ascorbic 2-phosphate (a stabilized vitamin C derivative), collagens and proteoglycans are produced, mimicking the native CSKs. Unfortunately, they do not proliferate in serum-free medium. Exposure to serum causes them to become fibroblastic, while TGFβ1 treatment resulted in a myofibroblastic phenotype (displaying stress fiber pattern).

Cell spheres formed from bovine CSKs in serum-free condition could maintain keratocyte expression features, however only 4-5% of native CSKs produce spheres (Scott et al., 2011). They underwent limited cell division and did not differentiate to myofibroblasts in response to TGFβ (Funderburgh et al., 2008). In contrast, mouse keratocytes in spheres could be propagated for 12 passages with the expression of keratocyte markers (Yoshida et al., 2005). Proliferation of primate CSKs was achieved by down-regulating TGFβ and receptor through promoter suppression in low calcium, serum-free condition (Kawakita et al., 2006). They expressed keratocan, CD34 and ALDH proteins. Human CSKs could maintain dendritic morphology and keratocan expression when cultured inside human amniotic membrane stroma, even in presence of serum (Espana et al., 2003). This could be due to the suppression of TGFβ/Smad signaling, which subsequently down-regulated αSMA and fibronectin (Tseng et al., 1999; Lee et al., 2000; Kawakita et al., 2005). Further evidence was provided by a reversal of myofibroblast to fibroblast phenotype when amniotic membrane stromal cells were seeded on amnion stromal matrix or in culture supplemented with amnion stromal extract (Li et al., 2008). Collectively, these studies have suggested that amnion stroma might contain soluble factors that are physiologically important in maintaining keratocyte phenotype and preventing myofibroblast differentiation. Nonetheless, growing cells in the opaque amniotic membrane stromal matrix is difficult for routine cell monitoring, for example cell viewing to examine cell growth status. Furthermore, amnion stroma contains its own stromal cells. Although they are sparsely located and should be destroyed by deep frozen storage, their remnants could affect keratocyte attachment and be a source of contaminants. Overall, maintaining corneal keratocytes in amnion stroma are mediated by the physical interaction between cells and stromal matrix substances as well as short-range chemokine reaction.

The identification of adult human corneal stromal stem cells (hCSSCs) offers the opportunity for the development of functional keratocytes through population doublings (Pinnamaneni et al., 2012). They produce stroma-like ECM components but they are yet to be organized globally to produce functional stromal tissues (Du et al., 2009; Wu et al., 2012). Lack of unique markers also makes the isolation of a homogenous and well-defined stem cell population difficult. In addition, human embryonic stem cell (hESC)-derived neural crest-like cells have also been induced to differentiate into keratocyte-like cells expressing keratocan and ALDH3A1 (Chan et al., 2013). However, the induction efficiency and cell purity are yet to be optimized.

Therefore, it is desirable to be able to cultivate human CSKs ex vivo to obtain an increased number of CSKs in a population which maintain their unique phenotypes as this is imperative for their future application in cell transplantation and therapy.

### SUMMARY OF THE INVENTION

The present invention relates to a method of cell culture utilising a dual culture medium protocol.

According to a first aspect, the present invention relates to a method for culturing corneal stromal keratocytes (CSKs) comprising:
(i) providing a population of CSKs comprising at least one corneal stromal keratocyte (CSK);
(ii) contacting the population of CSKs with a culture medium A supplemented with a liquid amnion extract and serum; and
(iii) replacing the culture medium A with a culture medium B supplemented with a liquid amnion extract or a minimum essential medium.

According to another aspect, the present invention relates to an isolated population of corneal stromal keratocytes (CSKs) obtainable/obtained by the method for culturing CSKs as described herein. The isolated population of CSKs comprises substantially of CSKs.

The invention also relates to a culture medium B supplemented with a liquid amnion extract.

Culture medium B may be further supplemented with at least one Rho-associated protein kinase inhibitor (ROCKi) and at least one insulin-like growth factor (IGF).

The invention further relates to a culture medium A supplemented with serum and a liquid amnion extract.

Culture medium A may also be further supplemented with at least one Rho-associated protein kinase inhibitor (ROCKi) and at least one insulin-like growth factor (IGF).

### BRIEF DESCRIPTION OF THE FIGURES

FIG. 1(A) shows the immunofluorescence results [phalloidin staining for stress fibres (horizontal rows 1 and 3), phallodin-AlexaFluor543 staining+lumican (horizontal rows 2 and 4). The supplements added are 0.5% FBS (vertical column 1 and rows 1-2); FBS+IGF1 (column 2 and rows 1-2); FBS+ROCKi (column 3 and rows 1-2); FBS+LAE (ASE) (5 μg/ml) (ASE=amnion stromal extract; FBS+ROCKi+LAE (ASE) (0.5 μg/ml) (column 1 and rows 3-4); FBS+ROCKi+LAE (ASE) (5 μg/ml) (column 2 and rows 3-4); FBS+ROCKi+IGF1+LAE (ASE) (5 μg/ml) (column 3 and rows 3-4) and ROCKi+IGF+LAE (ASE) (5 μg/ml) (column 4 and rows 3-4). (B) shows a plot of % cells with stress fiber pattern when cultured with KBM and different supplements.

FIG. 2 shows CSK morphology changes in different culture conditions for 96 hours using hanging drop method. (A) Primary human CSK remained as single round cells in serum-free KBM. (B) They displayed extended stromal cell-like morphology in serum-free ERI in KBM. (C) They formed spheroids in 0.5% FBS with ERI in KBM (KBM+0.5% SERI). (D) Cell quantification assay showing the percentages of cells with extended morphology and formation of aggregates, respectively.

FIG. 3 shows phase-contrast microscopic images of primary human CSKs cultured in KBM with (A) 2% FBS, (B) 0.5% FBS, (C) 0.5% FBS added with ERI cocktail (0.5% SERI) and (D) serum-free ERI condition, Inset in D shows cell-cell contacts between CSKs.

FIG. 4 shows the collagen gel contractibility of keratocytes. Human CSKs at passage 6 were seeded to collagen gel (2.5 mg/ml) under conditions (A) keratocyte basal medium (KBM) only; KBM with ((B) ERI, (C) 0.5% FBS, (D) 0.5% SERI, (E) 2% FBS and (F) 2% FBS with TGFβ1 (20 ng/ml). After 48 hours, the resulting gel area was quantified by Image J software and percentage of gel area to original well area was represented as median and interquartile range (IQR) in (G). *P<0.05 (Mann-Whitney U test) between indicated groups. Immunofluorescence of αSMA (red fluorescence) in gel was shown. Nuclei were stained with DAPI. Scale bar: 50 μm.

FIG. 5 shows CSKs from different animal species cultured in KBM+ERI cocktail with 0.5% FBS (columns 1 and 3) or serum-free (columns 2 and 4). All showed typical dendritic morphology.

FIG. 6 shows the pattern of Smad2/3 expression in human CSKs at passage 5 treated with TGFβ1 and LAE (ASE) fractions. (A) TGFβ1 (10 ng/ml) only; (B) TGFβ1 and LAE (ASE) (5 μg/ml); (C) KBM+ERI supplement and (D) keratocyte basal medium (KBM) only. After treatment for 3 days, the cells were fixed and immunolabeled for Smad2/3 and F-actin using phalloidin-FITC conjugate. (E) Quantification of cells showing nuclear localization of Smad2/3 after treatments for 3 days and 3 hours, respectively. The percentages of cells with nuclear Smad2/3 expression were presented as mean and SD from 10 fields of duplicated experiments. * indicating P<0.05 when compared to treatment with TGFβ1 only (one-way ANOVA with Dunn-Bonferroni correction). Scale bar: 50 μm.

FIG. 7 illustrates the immunofluorescence results of CSKs cultured under different conditions and shows that KBM+ERI reverted activated keratocytes to CSKs. (A) Immunofluorescence showed CSKs in KBM+0.5% SERI culture for up to 14 days had moderate cell proliferation without fibroblast transformation (negative alpha-SMA expression; column 4, row 2). The CSK-specific genes (ALDH1A1, keratocan, lumican) were down-regulated, compared to KBM+ERI (culture for 6 and 14 days (columns 1-3, row 2). This proved these cells to be “activated keratocytes”. When the cells were first cultured in KBM+0.5% SERI for 3 days, and then returned to KBM+serum-free ERI for another 3 days, all CSK genes re-expressed (columns 1-2, row 3). (B) Similar treatment of cells was performed for 14 days. The medium was switched at day 7. The cells re-expressed CSK-specific genes and were maintained as typical dendritic and stellate in morphology (column 3-4, row 3). This change was not seen when CSKs were cultured in KBM without ERI. CSKs cultured in KBM with 0.5% FBS but no ERI supplement for both 6 and 14 days did not express any keratocyte markers (columns 1-3, row 4). And Thy-1 express (indicating fibroblasts) even when cells returned to KBM+ERI condition.

FIG. 8 shows gene expression results. (A) Quantitative PCR showed the re-expression of CSK-specific genes when CSKs were returned to KBM in serum-free condition in the presence of ERI cocktail. The CSK genes (ALDH1A1, 3A1, keratocan, lumican and Col8A2) were down-regulated in KBM+0.5% SERI culture, compared to KBM+ERI culture for 6 and 14 days, respectively. With the minimal expression of Thy-1, this suggested these cells were “activated keratocytes”. When the cells first cultured in KBM+0.5% SERI for 3 days, then returned to serum-free KBM+ERI for another 3 days, all CSK genes re-expressed. Similar observation was found for cells in treatment for 14 days and the medium was switched at day 7. The general efficiency of retrieving CSK genes was higher than 60%. This change was not seen when CSKs were cultured in KBM without ERI cocktail. (B) CSKs in serum-free culture were generally quiescent whereas there was mild cell proliferation in KBM+0.5% SERI. (C) The retrieval of CSK genes was ERI-dependent. CSK cultured in KBM without ERI did not regain the gene expression and alpha-SMA remained expressed (indicating fibroblasts) even when cells were returned to KBM condition. On the other hand, α-SMA expression was suppressed when cells were changed from culture in KBM+0.5% FBS to KBM+serum-free ERI condition, however, the keratocyte genes (ALDH1A1 and ALDH3A1) were not retrieved.

FIG. 9 shows keratocan biosynthesis, expression and secretion in CSK cultured under different conditions. (A) Quantitative PCR analysis of the expression of keratocan and its biosynthesizing enzymes B3GNT7 and CHST6 in human CSKs at passage 5 under KBM+ERI, KBM+0.5% SERI and media switch (mo) conditions. Cells under KBM+serum (0.5% and 2%, respectively) culture served as controls. Keratocan expression in human cornea stromal tissue is represented as a bold horizontal line for the comparison with CSKs in culture. Data from quadruplicate experiments are presented as mean and SD. *P<0.05 (paired Student's t-test with Dunn-Bonferroni correction). (B) Western blot analysis of intracellular and extracellular keratocan expression in human and monkey CSKs under similar treatment as A. The intracellular protein samples were digested with endo-α-galactosidase before SDS-PAGE and western blotting for keratocan. β-Actin was used as housekeeping control. Band densitometry showed the changes of keratocan expression normalized with β-actin. Extracellular keratocan in monkey CSKs under similar treatment as A were detected by immunoprecipitation-western blot analysis. Paired conditioned medium samples digested with (+) or without (−) endo-α-galactosidase were screened for keratocan expression.

FIG. 10 depicts the keratocan expression of expanded human CSKs cultivated in bioscaffold. Left panel shows the immunofluorescence of keratocan expression in human CSKs (propagated in KBM+0.5% SERI until passage 6) and stromal fibroblasts (SF) (propagated in 0.5% FBS at passage 6) cultured in plastic compressed collagen for 3 weeks. Cytoplasmic keratocan expression was clearly observed in CSKs cultivated with KBM+0.5% SERI in compressed collagen matrix while not detectable in SF cultured with KBM+0.5% SERI and KBM+0.5% FBS, respectively. Right histogram shows the cell quantification analysis that the percentages of keratocan positive cells in two different human CSK cultures under KBM+0.5% SERI were significantly higher than SF cultures (close to 0%) (*P<0.05, multiple comparison using Kruskal-Wallis test and Dunn-Bonferroni correction).

FIG. 11 depicts the lumican expression of expanded human CSKs and stromal fibroblasts in bioscaffold. Left panel shows the immunofluorescence of lumican expression in human CSKs (propagated in KBM+0.5% SERI until passage 6) and stromal fibroblasts (SF) (propagated in KBM+0.5% FBS at passage 6) cultured in plastic compressed collagen for 3 weeks. Cytoplasmic lumican expression was observed in CSKs cultivated with KBM+0.5% SERI in compressed collagen matrix while not detectable in SF cultured with KBM+0.5% SERI and KBM+0.5% FBS, respectively. Right histogram shows the cell quantification analysis that the percentages of lumican positive cells in two different human CSK cultures under KBM+0.5% SERI were significantly higher than SF cultures (close to 0%) (*P<0.05, multiple comparison using Kruskal Wallis test and Dunn-Bonferroni correction).

FIG. 12 shows ALDH1A1 expression in expanded human CSKs and SFs in bioscaffold. Left panel shows the immunofluorescence detection of ALDH1A1 in expanded human CSK and SF under KBM+0.5% SERI while there is negligible staining in SF cultured with KBM+0.5% FBS (*P<0.05, multiple comparison using Kruskal Wallis test and Dunn-Bonferroni correction).

### DEFINITIONS

Amnion or amniotic membrane is the innermost layer of the placenta and is the first of two membranes (the other being the chorion or chorionic membrane) that surrounds the amniotic sac and consists of a thick basement membrane and an avascular stromal matrix.

Cell culture refers to the maintenance or growth of isolated cells in vitro, typically in an artificial environment. Cell culture includes cell expansion or propagation.

Cell culture expansion refers to cell culture where there is an increase in the number of cells. Cell expansion and cell propagation may be used interchangeably.

Cell culture substrate is used to mean a substrate upon which cells can live and/or grow. The substrate may be in the form of a culture vessel, for example a petri dish, flask, bottle, plate, tube, vial, etc, which can be welled or unwelled. Other substrates, such as two-dimensional or three-dimensional scaffolds, implants, microcarriers (e.g., beads composed of glass, plastic, or other materials), fiber beds, hollow fibers, stacked plate modules, or cell factories can also be utilized.

Cell passage refers to the splitting (dilution) and subsequent redistribution of a monolayer or cell suspension into culture vessels containing fresh medium. This is performed when the cells reached a desired level of density (for example ˜90%-full confluence).

The term passage number refers to the number of times that a cell population has been removed from the culture vessel and undergone a passage process.

In cell culture biology, confluence is the term commonly used as a measure of the coverage of the dish or the flask by the cells. For example, 100 percent confluence means the dish is completely covered by the cells, and therefore no more room is left for the cells to grow; whereas 50 percent confluence means roughly half of the dish is covered and there is still room for cells to grow.

As used herein the term “minimum essential medium” or “basal medium” refers to any serum-free culture medium of known composition which will support the viability of cells cultured in vitro.

As used herein, “serum-free” means that a medium does not contain serum or serum replacement, or that it contains essentially no serum or serum replacement. For example, an essentially serum-free medium can contain less than about 1%, 0.9%, 0.8%, 0.7%, 0.6%, 0.5%, 0.4%, 0.3%, 0.2% or 0.1% serum. As used herein, “serum replacement” refers to a composition added to a culture medium that mimics serum, but is typically not derived from animal products

### DETAILED DESCRIPTION OF THE INVENTION

The present invention relates to a method for culturing corneal stromal keratocytes (CSKs) comprising:
(i) providing a population of CSKs comprising at least one corneal stromal keratocyte (CSK);
(ii) contacting the population of CSKs with a culture medium A supplemented with a liquid amnion extract and serum; and
(iii) replacing the culture medium A with a culture medium B supplemented with a liquid amnion extract or a minimum essential medium.

The cell culture method may be a two-dimensional or three-dimensional cell culture method. Any suitable cell culture substrate may be used. The two-dimensional culture system or the three-dimensional culture method may be a large-scale, medium scale or small-scale method. The two-dimensional or three-dimensional culture method may be a batch culture method, a continuous culture method or a semi-continuous cell culture method.

A non-limiting example of a culture method is a hanging drop cell culture.

The CSKs may be contacted with culture medium A for any suitable period. During this period, the CSKs may be passaged for any number of times into culture medium A. The culture medium A may also be replaced with fresh culture medium A without passaging.

Replacing the culture medium A with the culture medium B or minimum essential medium includes removing the culture medium A and adding culture medium B or minimum essential medium respectively. Alternatively, the CSKs could be passaged from culture medium A into culture medium B or minimum essential medium respectively. Similarly, the CSKs could be contacted with culture medium B or minimum essential medium for any suitable period. During this period, the CSKs could be passaged for any number of times into culture medium B or minimum essential medium respectively. The culture medium B or minimum essential medium may also be replaced with fresh culture medium B or minimum essential medium, respectively without passaging.

It was surprisingly found that CSKs could expand in culture medium A with a proportion of CSKs developing into activated keratocytes but the activated keratocytes reverted back to CSKs in culture medium B or minimum essential medium.

The present invention relates to an isolated population of corneal stromal keratocytes (CSKs) obtainable/obtained by the method for culturing CSKs as described herein. The isolated population of CSKs comprises substantially of CSKs. It will be understood that the isolated population of CSKs may be used for any suitable purpose. For example, the CSKs may be used in further research. The CSKs may also be used for transplantation.

The invention also includes novel culture media for culturing CSKs. Accordingly, the invention also relates to a culture medium B supplemented with a liquid amnion extract.

The invention further relates to a culture medium A supplemented with a liquid amnion extract and serum.

The culture medium A may comprise any suitable culture medium supplemented with a liquid amnion extract and serum.

The culture medium B may also comprise any suitable culture medium supplemented with a liquid amnion extract.

Culture medium A and/or culture medium B may be further supplemented with at least one Rho associated protein kinase inhibitor (ROCKi) and/or at least one insulin-like growth factor (IGF).

Culture medium A and/or culture medium B may be further supplemented with at least one Rho associated protein kinase inhibitor (ROCKi) and at least one insulin-like growth factor (IGF).

The culture medium A and the culture medium B may comprise essentially the same components except that the culture medium A is supplemented with serum while the culture medium B is serum free or substantially serum-free.

The culture medium applicable for both culture medium A and culture medium B before supplementation with a liquid amnion extract, at least one Rho-associated protein kinase inhibitor (ROCKi) and at least one insulin-like growth factor (IGF) and serum if applicable may comprise any suitable culture medium. The suitable culture medium may be any minimum essential medium (MEM). Examples of suitable minimum essential medium include but are not limited to Eagle's minimum essential medium (Eagle's medium), a modified Eagle's medium [including but not limited to Dulbecco's Modified Eagle's Medium (DMEM) or Dulbecco's Modified Eagle's Medium: Nutrient Mixture F-12 (DMEM/F12)]. Commercially available DMEM/F12 may be used, including but not limited to DMEM/F12 from Invitrogen and Sigma-Aldrich. Culture medium A and culture medium B before supplementation with a liquid amnion extract, at least one Rho-associated protein kinase inhibitor (ROCKi) and at least one insulin-like growth factor (IGF) and serum if applicable may be the same culture medium or a different culture medium. Accordingly, culture medium A and culture medium B before supplementation with a liquid amnion extract, at least one Rho-associated protein kinase inhibitor (ROCKi) and at least one insulin-like growth factor (IGF) and serum if applicable may be the same minimum essential medium or a different minimum essential medium.

According to another aspect, the invention relates to a culture medium A comprising culture medium B further supplemented with serum.

Culture medium A and/or B may typically include other additional components. For example, the culture medium A, and/or B may be supplemented with at least one component selected from L-glutamate, 2-[4-(2-hydro xyethyl)piperazin-1-yl]ethanesulfonic acid (HEPES), insulin, transferrin, selenium, pyruvate, vitamins, amino acids, ascorbate and antibiotics. In particular, the culture medium comprises DMEM/F12 comprising 2 mM L-glutamate, 20 mM HEPES, 1% insulin-transferrin-selenium (Invitrogen), 1 mM sodium pyruvate 1% MEM Eagle's vitamin mixture (Lonza), 100 μM MEM non-essential amino acids (Invitrogen), 1 mM L-ascorbate 2-phosphate (Sigma) and antibiotics (for example: penicillin S, 100 U/ml and streptomycin sulphate, 100 μg/ml).

Any suitable amount of serum may be used to supplement the culture medium A. For example, the culture medium A may be supplemented with 0.1 to 10% serum. More suitably, the culture medium A may be supplemented with 0.5% or 2% serum. The serum for supplementing the culture medium A may be from any suitable source or may comprise serum replacement. For example, the serum includes but is not limited to bovine, equine, porcine, human serum. The serum may be from a fetal or an adult source. In particular, the serum may be fetal bovine serum (FBS). More in particular, the culture medium A may be supplemented with 0.5% or 2% FBS.

The liquid amnion extract may be derived from the fetal amnion from any mammal. For example, the liquid amnion extract could be bovine, equine, porcine, simian or human (non-exhaustive list). In particular, if the liquid amnion extract is human, it is derived from human placenta which would otherwise be medical waste. Any suitable amount of liquid amnion extract may be used to supplement culture medium A and/or culture medium B.

Any suitable Rho-associated protein kinase inhibitor (ROCKi) may be used. The ROCKi inhibitor includes a ROCK1 inhibitor, a ROCK2 inhibitor or an inhibitor of both ROCK1 and ROCK2. For example, the ROCKi includes but is not limited to (1R,4r)-4-((R)-1-aminoethyl)-N-(pyridin-4-yl)cyclohexanecarboxamide (Y-27632) and 5-(1,4-diazepane-1-sulfonyl)isoquinoline (Fasudil), N-benzyl-2-(pyrimidin-4-ylamino)thiazole-4-carboxamide (Thiazovivin), N-(6-fluoro-1H-indazol-5-yl)-2-methyl-6-oxo-4-(4-(trifluoromethyl)phenyl)-1,4,5,6-tetrahydropyridine-3-carboxamide (GSK429286A), 1-(3-Hydroxybenzyl)-3-(4-(pyridin-4-yl)thiazol-2-yl)urea (RKI-1447 or ROCK inhibitor XIII).

Any suitable amount of insulin-like growth factor may be used to supplement culture medium A and/or culture medium B. The insulin-like growth factor may be either insulin-like growth factor 1 (IGF1) or insulin-like growth factor 2 (IGF2).

Culture medium A and/or culture medium B [on its own or supplemented as herein described (liquid amnion extract, at least one Rho-associated protein kinase inhibitor (ROCKi); at least one insulin-like growth factor (IGF) and if applicable, serum, additional components as herein described and collagen] may be in the form of a liquid (e,g, an aqueous solution) or in the form of a solid or semi-solid (for e,g, a gel).

The method for culturing CSKs may be carried out in the presence of collagen. The collagen may be coated on the cell culture substrate. Alternatively, culture medium B and/or culture medium A according to any according to any aspect of the invention described herein may be further supplemented with collagen.

The invention also includes a kit or a combination comprising culture medium A and/or culture medium B according to any aspect of the invention.

The invention also includes a kit or a combination comprising culture medium A, culture medium B and serum.

The invention also includes a kit or a combination comprising culture medium A and a minimum essential medium. The invention also includes a kit or a combination comprising culture medium A, a minimum essential medium and serum.

The invention also includes a kit or a combination comprising culture medium B and serum. The invention also includes a kit or a combination comprising culture medium B, a minimum essential medium and serum.

Including serum in the kit or combination enables the user to increase the amount of serum in the minimum essential medium or culture medium B, as appropriate.

The culture medium A, culture medium B, minimum essential medium and/or serum may be dispensed in separate containers.

Having now generally described the invention, the same will be more readily understood through reference to the following examples which are provided by way of illustration, and are not intended to be limiting of the present invention.

### EXAMPLES

### Example 1

### Materials and Methods

### Corneal Stromal Tissue

Research grade human cadaveric cornea tissues were obtained from Lions Eye Institute for Transplant and Research Inc. (Tampa, Fla., US). In addition, transplant grade human cadaveric corneoscleral tissues after transplantation were obtained from Singapore Eye Bank, Singapore National Eye Centre (Singapore), with consent for research use. The human corneoscleral specimens with endothelial cell count greater than 2,000 cells per mm² were procured, and preserved in Optisol-GS at 4° C. and transported to the culture laboratory within 14 days of preservation.

### Isolation and Culture of Human Corneal Stromal Keratocytes

Cornea specimens were washed in sterile PBS (0.01 M, Invitrogen, Carlsbad, Calif., US) added with 3% antibiotics-antimycotes (penicillin S, streptomysin sulfate and amphoptericin B, Invitrogen). Central corneal buttons (1 mm from peripheral limbus) were trephined and treated with dispase II (20 mg/ml, Roche, Basal, Switzerland) to remove corneal epithelium and endothelium. The stroma tissue was trimmed into small fragments (˜1 mm³ in size) and digested with collagenase I (0.1%, Worthington, Lakewood, N.J., US) in keratocyte basal medium (KBM) for 8 to 10 hours at 37° C. After repeat pipetting, the cell suspension was passed through cell strainer (40 μm pore size), followed by centrifugation at 400 g for 5 min at room temperature. The cell pellet was washed and suspended in keratocyte basal medium (KBM), which is DMEM/F12 medium (Invitrogen) supplemented with 2 mM L-glutamate, 20 mM HEPES, 1% insulin-transferrin-selenium (Invitrogen), 1 mM sodium pyruvate, 1% MEM Eagle's vitamin mixture (Lonza), 100 μM MEM non-essential amino acids (Invitrogen), 1 mM L-ascorbate 2-phosphate (Sigma) and antibiotics (penicillin S, 100 U/ml and streptomycin sulphate, 100 μg/ml). Cells were seeded at 10⁴ cells per cm² on collagen I coated culture wells (BD Biosciences, Franklin Lakes, N.J., US). To propagate genuine CSKs, the cells must meet three basic requirements: (1) has dendritic morphology, (2) absence of fibroblast features (negative expression of αSMA and F-actin stress fiber pattern) and (3) express CSK markers (e.g. ALDH1A1, ALDH3A1, lumican, keratocan and COL8A2). Cells were cultured in the presence of various chemicals and growth factors, including liquid amnion extract (LAE) or soluble amnion stromal extract (ASE), preparation details as shown below), ROCK inhibitor Y27632 (10 Millipore, Billerica, Mass., US), insulin-like growth factor 1 (10 ng/ml, IGF1, Invitrogen) and/or fetal bovine serum (FBS, Invitrogen). Medium change was performed every 3 days. When cells reached about 70% confluence, they were detached with TryPLE Express (Invitrogen) for 5 to 10 minutes and plated to new culture surface under the same seeding density. Cells not exceeding passage 6 were used in the experiments.

### Preparation of Liquid Amnion Extract (LAE) or Soluble Amnion Stromal Extract (ASE)

Fresh human fetal amnion was isolated from placenta from a postpartum female younger than 40 years old (following cesarean section. Written consent was obtained under an institutional review board-approved protocol. After extensive rinses with sterile saline to remove all blood traces, amnion was manually peeled from the chorion. The proximal amniotic membrane (AM) from the proximal one-fourth to the distal one-third to the placental disc was taken for LAE preparation. The LAE is predominantly amnion stromal extract (ASE) and the terms LAE and ASE may be used interchangeably. The tissue was rinsed with PBS added with antibiotics (for example penicillin S, 100 U/ml and streptomycin sulphate, 100 μg/ml) and antimycotes and cut into pieces (4×4 cm² in size) and frozen in 50% glycerol in DMEM (Invitrogen) for 1 week at −80° C. The amnion is thus devitalised as living cells are typically destroyed during freezing. The sample was thawed and rinsed twice in PBS. The amnion pieces were briefly drip-dried, weighed, grounded under the air phase of liquid nitrogen to homogenate and suspended in ice-cold sterile PBS (5 ml per gram tissue). The mixture was rotated at 300 rpm for 48 hours at 4° C. The suspension was centrifuged at 14,000 g for 20 min at 4° C. to remove debris. The clear supernatant was further centrifuged in a Centrifugal Filters (UltraCel-3K, Amicon, Millipore) for 4000 g for 60 min at room temperature. The concentrated solute was collected and stored in aliquots at −80° C. The total protein concentration of LAE (ASE) was determined by Protein DC assay (BioRad) and TIMP1 content was measured by human TIMP1 enzyme-linked immunosorbant assay (ELISA) kit (Invitrogen).

### Protein Characterization of Soluble LAE (ASE) by One-Dimensional Nano-Scale Liquid Chromatography Coupled to Tandem Mass Spectrometry (Nano LC-MS/MS)

LAE (ASE) samples (100 μg) were denatured with Tris-HCl (0.1 M), SDS (2%) and Tris-(2-carboxyethyl) phosphine (TCEP, 33 μM, Sigma) at 60° C. for 1 hour, followed by washing with urea (1 M, Sigma) and blocking with iodoacetamide (Sigma) in urea in a centrifugal filter unit. The sample was collected by spinning at 14,000 g for 10 minutes, washed with urea and equilibrated with ammonium bicarbonate (50 mM, Sigma) before trypsin digestion. After washes, the digested sample was eluted and trypsin was quenched by formic acid (10%, Sigma). It was then desalted with Silica C-18 ultra-microspin column (Nestgroup, Southburough, Mass., US) and analyzed by one-dimensional nano LC-MS/MS using Dionex UltiMate 3000 (ThermoFisher Scientific, Waltham, Mass., US) coupled with AB Sciex Triple TOF 5600. The sample was loaded to the Acclaim PepMap trap column (ThermoFisher) and directed at a flow rate of 5 μl/min to Acclaim PepMap RSLC column (ThermoFisher), which was connected to a spray tip (PicoTip Emitter Silica Tip™, New Objective, Woburn, Mass., US). The total gradient time was set at 120 minutes. Mobile phase A was formic acid (0.1%) and acetonitrile (2%) while B was formic acid (0.1%) and acetonitrile (98%). To create the separation gradient for eluting peptides, mixtures with an increasing concentration of B were prepared as follows: 5 to 7% for 12 minutes; 7 to 24% for 57 minutes; 24 to 40% for 27 minutes; 40 to 60% for 7 minutes; 60 to 95% for 1 minute; and the column was equilibrated at 95% for 16 minutes. The experimental parameters for mass spectrometer were: Ionspray Voltage Floating at 2.4 kV, Curtain Gas at 30, Ion Source Gas 1 at 12, Interface Heater at 125° C., Declustering Potential at 100V and Nebuliser Current for N₂ at 3. Data were collected by TripleTOF5600 system using Analyst TF 1.6 software by AB Sciex in the Information-dependent acquisition (IDA) mode. Peptides were profiled under 350 to 1250 Da mass range, and a MS/MS product ion scan from 100 to 1500 Da, with the abundance threshold set at 120 counts per second and accumulation time for ions at 50 msec. Target ions were excluded from the scan for 12 sec after detection and former ions were excluded from the scan after one repetition. The IDA advanced ‘rolling collision energy (CE)’ option was set to automatically ramp up the CE value in the collision cell as the m/z value was increased. A maximum of 30 MS/MS spectra was collected from candidate ions per cycle.

### Proteomics Data Processing and Pathway Analysis

MS/MS data output was analyzed by ProteinPilot software ver 4.5 (AB SCIEX, US) with the search against International Protein Index (IPI) human protein database vers 3.77 to identified candidate proteins. “Reversed Protein Sequences” was set for the ProteomicS Performance Evaluation Pipeline (PSPEP) software (AB SCIEX, US). The Paragon™ search algorithm in ProteinPilot was configured as: (1) Sample type: identification, (2) Cys alkylation: iodoacetamide, (3) Digestion: trypsin, (4) Instrument: TripleTOF 5600, (5) Special factors: none, (6) ID Focus: Biological modifications and (7) Search effort: Thorough ID and 95% confidence level was used. False Discovery Rate (FDR) analysis in the ProteinPilot software was performed and FDR <1% was set for protein identification. Reverse database search strategy was used to calculate FDR for peptide identification. Pathway analysis was done by MetaCore™ software (GeneGO, San Diego, Calif., US). “Pathway Maps” were generated with P<10⁻⁴ representing the pathways significantly enriched with the identified proteins.

### Hanging Drop Cell Culture

Fresh human CSKs were suspended in 100 μl of the respective medium. Drops (10 volume) were deposited to the inner side of lid of a cell culture dish (60 mm diameter), which was added with 5 ml PBS. The lid was placed back to the culture dish and the setup was kept in 37° C. culture incubator for 96 hours. Under stereomicroscope, the cell sheet or aggregate formation was monitored. The amount of flattened cells or aggregates was quantified in a minimum of 10 drops and the mean percentages were compared with the significance calculated by paired Student's t-test and adjusted for type I error P value using Dunn-Bonferroni post-hoc test.

### Immunofluorescence

The samples were fixed with freshly prepared 2% neutral buffered paraformaldehyde (Sigma), quenched with ice-cold 50 mM ammonium chloride (Sigma) and permeabilized with 0.15% saponin (Sigma). After blocking with 2% bovine serum albumin (Sigma) and 2% normal goat serum (Invitrogen), the cells were incubated with primary antibody recognizing aldehyde dehydrogenase 1A1 (ALDH1A1, Proteintech), lumican (LUM, Bioss), transketolase (Tkt, Cell Signalling), αSMA (Sigma) or Alexa Fluor 543-conjugated phalloidin (Sigma), respectively. The secondary antibodies were either Alexa488 or Rhodamine Red-X-conjugated (Jackson ImmunoRes Lab, West Grove, Pa.). All nuclei were counterstained with DAPI (4′,6-diamidino-2-phenylindole, Santa Cruz) and samples were mounted in FluorShield (Sigma). Results were visualized under fluorescence microscopy (Carl Zeiss) or confocal laser-scanning microscopy (LSM 510 Meta, Carl Zeiss).

From Phalloidin staining, cells displaying stress fiber pattern were quantified from a minimum of 10 fields viewed at 20× magnification (objective). The percentage of stress cells in triplicate experiments was expressed as mean±SD. Results were compared and analyzed by paired Student's t-test and Dunn-Bonferroni correction.

### Gene Expression and Polymerase Chain Reaction

Cells were collected in RLT (guanidine thiocyanate) buffer (Qiagen) freshly added with 1% β-mercaptoethanol (Sigma) and total RNA was extracted by RNeasy kit (Qiagen, Valencia, Calif., US) and on-column RNase-free DNase kit (Qiagen) according to manufacturer's protocols. Reverse transcription of 1 μg total RNA was performed with Superscript III RT-PCR kit (Invitrogen) using random hexanucleotide primer (10 ng/ml, Invitrogen). Gene expression was assayed by quantitative real-time PCR (qPCR) using Sybr Green Supermix (BioRad) in GFX96 Real-time System (BioRad). Experiments were run in quadruplicate. Relative gene expression of each sample was normalized by the mean CT value to housekeeping β-actin (ACTB) and fold changes to human stromal tissue or untreated cells were calculated.

### Collagen Gel Contraction Assay

Bovine type I collagen gels were made to a final concentration of 2.5 mg/ml as liquid with KBM and supplements. It was mixed with cells at a density of 10⁵ cells/ml and 0.5 ml mixture was added to each well of 24-multiwell plate coated with 1% BSA. It was allowed to gelation at 37° C. for 1 hour before addition of appropriate medium. After 24 hours, the gel was released from the side of culture well with a sterile needle to initiate contraction. The collagen gel size change was recorded at 48-hour interval and measured using Image J software (NIH). The percentages of collagen gel size to the size of culture well were compared among different treatments. Experiment was done in quadruplicate and the percentages of gel contraction were represented as median and interquartile range. Intergroup significance was calculated by Mann-Whitney U test.

Culture to Revert CSKs from “Activated Keratocytes”

Human CSKs were cultured in KBM supplemented with LAE (ASE), ROCK inhibitor (Y27632), IGF1 and 0.5% FBS (termed as KBM+0.5% SERI) for 3 or 7 days, followed by replenishing with medium of the same formulation (KBM) except for FBS (termed as KBM+serum-free ERI or KBM+ERI) for another 3 or 7 days, respectively. Cells cultured uninterruptedly in KBM+ERI, KBM+0.5% SERI or KBM added with FBS served as controls. Fresh medium was replenished every 3 or 4 days. At day 6 or 14, cells and conditioned media were collected for immunofluorescence, RNA and western blot analyses.

### Keratocan Expression

Keratocan secretion was detected in culture medium conditioned by human CSK culture under KBM+ERI protocol. The medium was collected and spun to remove cell debris. In addition, intracellular keratocan was assayed in the expanded CSKs. The cells were suspended at 10⁵ cells/ml in PBS added with 0.5% Triton X-100 (Sigma) on ice for 20 minutes. After spinning at 25,000 g for 15 min at 4° C., Triton X-100 insoluble fraction was collected and was further extracted in buffer containing 4 M guanidine-HCl (Sigma), 10 mM sodium acetate (Sigma), 10 mM disodium EDTA (Sigma), 5 mM aminobenzamidine (Sigma) and 0.1 M α-amino-n-caproic acid (Sigma), pH 7.2. Both samples were concentrated through an Amicon™ Ultra Centrifugal Filter (3 k cut-off, Millipore) at 14,000 g for 20 min at 4° C. Proteins were recovered in 0.1 M Tris acetate buffer (pH 6.0, Sigma) with 6 M urea and the protein concentration was quantified at OD₂₈₀. Protein aliquots (100 μg) were biotinylated using EZ-linked Sulfo-NHS-Biotinylation kit (Thermo Scientific, Waltham, Mass., US) under manufacturer's instruction. Briefly, 11 mM Sulfo-NHS-biotin in PBS was added in a 20-fold molar excess to sample proteins and incubated for 1 hr at room temperature with rotation. The mixture was then allowed to absorb in a pre-washed Zeba desalt spin column (Thermo Scientific) for 10 min and then centrifuged. The flow-through with biotinylated proteins was divided into 2 fractions. One was treated with 0.1 U/ml endo-3-galactosidase (Sigma) in phosphate buffer (pH 5.8) for 1 hr at 37° C. and the untreated half was left on ice. Both fractions were immuno-precipitated with Protein A-conjugated magnetic beads (Millipore) pre-bound with polyclonal antibody against keratocan (Sigma). After magnetic separation and washes in PBS, the beads were denatured in 50 mM Tris-HCl (pH 6.8) added with 1% SDS and 0.25 M f3-mercaptoethanol at 95° C.). The protein sample was then resolved using gradient SDS-PAGE (4-20%) and western blotted with streptavidin-horseradish peroxidase conjugated (Thermo Scientific). Signal was detected with enhanced chemiluminescense (ECL, BioRad). Band intensity was analyzed by Quantity One Imaging software (BioRad) and the intracellular expression of keratocan was normalized with that of β-actin for comparison.

### Plastic Compressed Collagen Gel Culture

The preparation of plastic compressed collagen gel was adapted from the protocol described in Levis et al. (2012). Collagen solution containing 80% vol/vol sterile rat-tail type I collagen (2.06 mg/ml, First Link Ltd., Bath, UK) in 10% of 10× (KBM+0.5% SERI) or 10× (KBM+0.5% FBS) with neutralization by 1 N sodium hydroxide (Sigma). The solution was then mixed on ice with 10% vol/vol cell suspension to obtain a final density of 100,000 cells/ml. The cell/collagen mixture was left on ice for 30 minutes to get rid of air bubbles while preventing gelling. The solution was then casted in 24-well plate with a volume of 1.5 ml per well and allowed to form collagen gel at 37 C for 30 min. The set gel was then subjected to a confined compression using absorbent plunger for 15 minutes at room temperature. The thin collagen construct (RAFT) was then immediately nourished with the respective medium (either KBM+0.5% SERI or KBM+0.5% FBS). Medium was replenished every 3 days. The culture was maintained for 3 weeks. RAFT constructs were fixed with neutral buffered 2% paraformaldehyde for 30 minutes at 37° C. down to room temperature and stored in PBS added with 0.1% paraformaldehyde until immunofluorescence for keratocyte markers (keratocan, lumican and ALDHIA1).

### Example 2

### Results and Discussion

### LAE (ASE) and Protein Characterization

Soluble LAE (ASE) was prepared from frozen AM collected from the proximal one-fourth to the distal one-third to the placental disc. The devitalized AM was grounded to homogenate and extracted with ice-cold PBS. After removing debris by high-speed centrifugation, the clear supernatant was concentrated by spinning in UltraCel-3K. The protein profile of was successfully mapped with peptide homology ≧95% from the database of 178,828 proteins (Table 1). The candidate protein list was validated by choosing to measure TIMP1 level using ELISA and the concentration was 6.4±4.7 ng per μg protein. Furthermore, significant pathway analysis by MetaCore™ predicted that these proteins could participate in 12 major pathways (P<10⁻⁴ and False Discovery Rate FDR<10⁻³). They included TGFβ signaling, cytoskeleton and ECM remodeling, protein folding and maturation as well as immune responses (Table 2).

TABLE 1
Protein list from LAE (ASE) by nanoLC-MS/MS analysis

| | % | | | Peptides |
|---|---|---|---|---|
| | Coverage | IPI Accession # | Name | (95%) |
| 1 | 77.4 | IP00021812.2 | AHNAK/Desmoyokin Neuroblast differentiation- | 362 |
| | | | associated protein | |
| 3 | 30.5 | IPI00946793.1 | TNXB 458 kDa Tenascin XB protein | 75 |
| 4 | 68.1 | IPI00745872.2 | ALB Isoform 1 of serum albumin | 90 |
| 5 | 40.8 | IPI00339224.2 | FN1 fibronectin isoform 4 preproprotein | 58 |
| 6 | 55.7 | IPI00021405.3 | LMNA Isoform A of prelamin-A/C | 53 |
| 7 | 62 | IPI00418471.6 | VIM Vimentin | 42 |
| 8 | 25.6 | IPI00943563.1 | FLNB Isoform 2 of filamin-B | 37 |
| 9 | 74.6 | IPI00455315.4 | ANXA2 Isoform 1 of annexin A2 | 52 |
| 10 | 52.8 | IPI00003362.2 | HSPA5 HSPA5 protein | 37 |
| 11 | 55.2 | IPI00022463.1 | TF Serotransferrin | 34 |
| 12 | 39.2 | IPI00029739.5 | CFH Isoform 1 of complement factor H | 30 |
| 13 | 31.3 | IPI00419215.6 | A2ML1 Alpha-2-macroglobulin-like protein 1 | 28 |
| 14 | 55.1 | IPI00876888.1 | LOC100294459; IGHG1; LOC100290146; IGHV4-31 | 45 |
| | | | FLJ78387 | |
| 15 | 60.1 | IPI00304925.5 | HSPA1B; HSPA1A Heat shock 70 kDa protein 1A/1B | 33 |
| 16 | 56.2 | IPI00025252.1 | PDIA3 Protein disulfide-isomerase A3 | 25 |
| 17 | 56 | IPI00944977.1 | ALCAM, FLJ79012, highly similar to CD166 antigen | 25 |
| 18 | 62.3 | IPI00479145.3 | KRT19 Keratin, type I cytoskeletal 19 | 25 |
| 19 | 21.2 | IPI00220213.2 | TNC Isoform 4 of tenascin | 23 |
| 20 | 53.4 | IPI00554648.3 | KRT8 Keratin, type II cytoskeletal 8 | 24 |
| 21 | 23.1 | IPI00856098.1 | RRBP1 p180/ribosome receptor | 22 |
| 22 | 70.5 | IPI00465248.5 | ENO1 Isoform alpha-enolase | 22 |
| 23 | 44.9 | IPI00009865.4 | KRT10 Keratin, type I cytoskeletal 10 | 25 |
| 24 | 34.8 | IPI00953666.1 | ABP1 Primary amine oxidase | 20 |
| 25 | 59.3 | IPI00872379.1 | ANXA5 36 kDa protein | 21 |
| 26 | 30.4 | IPI00220327.4 | KRT1 Keratin, type II cytoskeletal 1 | 24 |
| 27 | 49.6 | IPI00947127.1 | LDHA L-lactate dehydrogenase A chain isoform 3 | 20 |
| 28 | 52.9 | IPI00012119.1 | DCN Isoform A of decorin | 25 |
| 29 | 36.9 | IPI00329108.4 | SCEL Isoform 1 of sciellin | 17 |
| 30 | 53.1 | IPI00970915.1 | CAST Isoform 3 of calpastatin | 18 |
| 31 | 71.6 | IPI00219018.7 | GAPDH Glyceraldehyde-3-phosphate dehydrogenase | 22 |
| 32 | 60.5 | IPI00872780.2 | ANXA4 FLJ51794, highly similar to annexin A4 | 19 |
| 33 | 13.5 | IPI00880007.2 | MAP4 245 kDa protein | 16 |
| 34 | 17.5 | IPI00478003.3 | A2M Alpha-2-macroglobulin | 16 |
| 35 | 50 | IPI00553177.1 | SERPINA1 Isoform 1 of alpha-1-antitrypsin | 17 |
| 36 | 56.1 | IPI00010779.4 | TPM4 Isoform 1 of tropomyosin alpha-4 chain | 15 |
| 37 | 53.7 | IPI00646304.4 | PPIB Peptidyl-prolyl cis-trans isomerase B | 18 |
| 38 | 59.2 | IPI00020986.2 | LUM Lumican | 21 |
| 39 | 22.4 | IPI00292530.1 | ITIH1 Inter-alpha-trypsin inhibitor heavy chain H1 | 15 |
| 40 | 48 | IPI00218918.5 | ANXA1 Annexin A1 | 13 |
| 41 | 54.9 | IPI00169383.3 | PGK1 Phosphoglycerate kinase 1 | 15 |
| 42 | 16.7 | IPI00296099.6 | THBS1 Thrombospondin-1 | 14 |
| 43 | 58.5 | IPI00020599.1 | CALR Calreticulin | 14 |
| 44 | 41.2 | IPI00006114.4 | SERPINF1 Pigment epithelium-derived factor | 13 |
| 45 | 56.5 | IPI00941900.1 | CALU Isoform 1 of calumenin | 12 |
| 46 | 34.7 | IPI00019359.4 | KRT9 Keratin, type I cytoskeletal 9 | 14 |
| 47 | 54 | IPI00784985.1 | IGKV3-20; IGK@ protein | 19 |
| 48 | 43.2 | IPI00759596.1 | HNRNPC Isoform 4 of Heterogeneous nuclear | 13 |
| | | | ribonucleoproteins C1/C2 | |
| 49 | 50.2 | IPI00021263.3 | YWHAZ 14-3-3 protein zeta/delta | 12 |
| 50 | 5.3 | IPI00215631.1 | VCAN Isoform Vint of versican core protein | 12 |
| 51 | 78.9 | IPI00654755.3 | HBB Hemoglobin subunit beta | 11 |
| 52 | 29.1 | IPI00010796.1 | P4HB Protein disulfide-isomerase | 11 |
| 53 | 31.9 | IPI00021304.1 | KRT2 Keratin, type II cytoskeletal 2 epidermal | 16 |
| 54 | 54.8 | IPI00930226.1 | ACTG1 FLJ57283, highly similar to actin, cytoplasmic 2 | 13 |
| 55 | 25.8 | IPI00604620.3 | NCL Nucleolin | 11 |
| 56 | 26.7 | IPI00303476.1 | ATP5B ATP synthase subunit beta, mitochondrial | 10 |
| 57 | 65.1 | IPI00797270.4 | TPI1; TPI1P1 Isoform 1 of triosephosphate isomerase | 11 |
| 58 | 31.9 | IPI00003865.1 | HSPA8 Isoform 1 of Heat shock cognate 71 kDa protein | 19 |
| 59 | 66.7 | IPI00419585.9 | PPIA Peptidyl-prolyl cis-trans isomerase A | 12 |
| 60 | 51.1 | IPI00848226.1 | GNB2L1 Guanine nucleotide-binding protein subunit | 9 |
| | | | beta-2-like 1 | |
| 61 | 37.1 | IPI00025465.2 | OGN cDNA FLJ59205, highly similar to mimecan | 10 |
| 62 | 32.8 | IPI00916517.1 | HNRNPA2B1 Uncharacterized protein | 9 |
| 63 | 47 | IPI00426051.3 | IGHG2 Putative uncharacterized protein | 31 |
| | | | DKFZp686C15213 | |
| 64 | 26.2 | IPI00647837.5 | ZNF185 Isoform 3 of Zinc finger protein 185 | 10 |
| 65 | 22.3 | IPI00021033.2 | COL3A1 Isoform 1 of collagen alpha-1(III) chain | 24 |
| 66 | 25.1 | IPI00297550.8 | F13A1 Coagulation factor XIII A chain | 8 |
| 67 | 30.3 | IPI00514023.5 | PTRF FLJ53495, highly similar to polymerase I and | 10 |
| | | | transcript release factor | |
| 68 | 19.2 | IPI00965868.1 | TGFBI Transforming growth factor, beta-induced, 68 kDa | 9 |
| | | | variant | |
| 69 | 25.3 | IPI00022488.1 | HPX Hemopexin | 8 |
| 70 | 31.8 | IPI00796333.1 | ALDOA 45 kDa protein | 8 |
| 71 | 85.2 | IPI00008529.1 | RPLP2 60S acidic ribosomal protein P2 | 8 |
| 72 | 25.6 | IPI00016801.1 | GLUD1 Glutamate dehydrogenase 1, mitochondrial | 8 |
| 73 | 15.8 | IPI00790739.1 | ACO2 Aconitase 2, mitochondrial | 8 |
| 74 | 11.5 | IPI00939199.1 | ABI3BP 115 kDa protein | 8 |
| 75 | 24.8 | IPI00943074.1 | PRKCSH Glucosidase 2 subunit beta | 8 |
| 76 | 56.1 | IPI00479185.1 | TPM3 tropomyosin alpha-3 chain isoform 4 | 15 |
| 77 | 43.2 | IPI00306413.3 | TPPP3 Tubulin polymerization-promoting protein family | 7 |
| | | | member 3 | |
| 78 | 37.5 | IPI00295741.4 | CTSB Cathepsin B | 7 |
| 79 | 26 | IPI00217467.3 | HIST1H1E Histone H1.4 | 7 |
| 80 | 44.1 | IPI00647915.1 | TAGLN2 24 kDa protein | 8 |
| 81 | 32.7 | IPI00022391.1 | APCS Serum amyloid P-component | 7 |
| 82 | 35.6 | IPI00969620.2 | IGLC3; IGLC1; IGLV1-44; IGLL5 hypothetical protein | 8 |
| | | | XP_002348153 | |
| 83 | 59.3 | IPI00759663.1 | PRDX5 Isoform cytoplasmic + peroxisomal of | 7 |
| | | | peroxiredoxin-5, mitochondrial | |
| 84 | 12.4 | IPI00645038.1 | ITIH2 Inter-alpha (globulin) inhibitor H2 | 7 |
| 85 | 6.1 | IPI00783987.2 | C3 Complement C3 (Fragment) | 6 |
| 86 | 34.2 | IPI00027827.2 | SOD3 Extracellular superoxide dismutase [Cu—Zn] | 6 |
| 87 | 41.2 | IPI00000816.1 | YWHAE 14-3-3 protein epsilon | 10 |
| 88 | 27.5 | IPI00847322.1 | SOD2 superoxide dismutase 2, mitochondrial isoform A | 6 |
| | | | precursor | |
| 89 | 38 | IPI00794894.1 | SNORD4A; RPL23A protein | 6 |
| 90 | 46.8 | IPI00641249.2 | ATP5A1 18 kDa protein | 6 |
| 91 | 39.6 | IPI00465431.8 | LGALS3 Galectin-3 | 7 |
| 92 | 38.6 | IPI00640741.1 | PRDX1 19 kDa protein | 7 |
| 93 | 5.4 | IPI00013933.2 | DSP Isoform DPI of desmoplakin | 6 |
| 94 | 35 | IPI00412579.6 | RPL10A 60S ribosomal protein L10a | 6 |
| 95 | 41.9 | IPI00921977.1 | TPM1 FLJ54760, highly similar to tropomyosin 1, alpha | 11 |
| 96 | 40.8 | IPI00549248.4 | NPM1 Isoform 1 of nucleophosmin | 7 |
| 97 | 20.9 | IPI00449920.1 | IGHA1 FLJ90170 fis, highly similar to Ig alpha-1 chain C | 7 |
| | | | region | |
| 98 | 28.8 | IPI00021841.1 | APOA1 Apolipoprotein A-I | 5 |
| 99 | 41.1 | IPI00009028.1 | CLEC3B Tetranectin | 6 |
| 100 | 30.8 | IPI00965952.1 | HNRNPD Uncharacterized protein | 5 |
| 101 | 26.1 | IPI00877792.1 | FGG Uncharacterized protein | 6 |
| 102 | 23 | IPI00297646.5 | COL1A1 Collagen alpha-1(I) chain | 15 |
| 103 | 46.4 | IPI00183695.9 | S100A10 Protein S100-A10 | 7 |
| 104 | 63.4 | IPI00410714.5 | HBA1; HBA2 Hemoglobin subunit alpha | 7 |
| 105 | 64 | IPI00939544.2 | HBG2 A-gamma globin Osilo variant | 7 |
| 106 | 27 | IPI00939124.1 | HNRNPA3 37 kDa protein | 7 |
| 107 | 18.8 | IPI00018206.4 | GOT2 Aspartate aminotransferase, mitochondrial | 5 |
| 108 | 38.4 | IPI00411765.3 | SFN Isoform 2 of 14-3-3 protein sigma | 8 |
| 109 | 12.5 | IPI00952947.1 | MSLN Uncharacterized protein | 5 |
| 110 | 31.4 | IPI00384444.6 | KRT14 Keratin, type I cytoskeletal 14 | 13 |
| 111 | 3 | IPI00964552.1 | COL12A1 Uncharacterized protein | 5 |
| 112 | 15 | IPI00220503.9 | DCTN2 dynactin subunit 2 | 5 |
| 113 | 26.7 | IPI00967849.1 | C1QB Uncharacterized protein | 5 |
| 114 | 28.8 | IPI00641737.1 | HP Haptoglobin | 5 |
| 115 | 34.5 | IPI00215780.5 | RPS19 40S ribosomal protein S19 | 5 |
| 116 | 41.3 | IPI00011937.1 | PRDX4 Peroxiredoxin-4 | 5 |
| 117 | 21.9 | IPI00853455.1 | CTSD protein | 5 |
| 118 | 14 | IPI00847635.1 | SERPINA3 Isoform 1 of alpha-1-antichymotrypsin | 5 |
| 119 | 8.2 | IPI00947307.1 | CP cDNA FLJ58075, highly similar to ceruloplasmin | 5 |
| 120 | 14.6 | IPI00032220.3 | AGT Angiotensinogen | 5 |
| 121 | 38.1 | IPI00013895.1 | S100A11 Protein S100-A11 | 6 |
| 122 | 52.9 | IPI00220362.5 | HSPE1 10 kDa heat shock protein, mitochondrial | 4 |
| 123 | 17.7 | IPI00304692.1 | RBMX Heterogeneous nuclear ribonucleoprotein G | 4 |
| 124 | 28.3 | IPI00930599.1 | MYL6B 16 kDa protein | 4 |
| 125 | 19.3 | IPI00060423.4 | CTHRCI Isoform 1 of Collagen triple helix repeat- | 4 |
| | | | containing protein 1 | |
| 126 | 21.2 | IPI00022394.2 | C1QC Complement C1q subcomponent subunit C | 4 |
| 127 | 57 | IPI00219219.3 | LGALS1 Galectin-1 | 4 |
| 128 | 14.5 | IPI00014516.1 | CALD1 Isoform 1 of caldesmon | 4 |
| 129 | 5.6 | IPI00909030.1 | KTN1 cDNA FLJ61494, highly similar to kinectin | 4 |
| 130 | 4.8 | IPI00384542.4 | NID1 Isoform 2 of Nidogen-1 | 4 |
| 131 | 44.6 | IPI00032313.1 | S100A4 Protein S100-A4 | 6 |
| 132 | 10.6 | IPI00946337.1 | IGHM 52 kDa protein | 4 |
| 133 | 37.8 | IPI00442073.5 | SRP1 Cysteine and glycine-rich protein 1 | 4 |
| 134 | 9.6 | IPI00879931.1 | SERPING1 FLJ58826, highly similar to plasma protease | 4 |
| | | | C1 inhibitor | |
| 135 | 2.9 | IPI00292150.5 | LTBP2 Latent-transforming growth factor beta-binding | 4 |
| | | | protein 2 | |
| 136 | 63.6 | IPI00013917.3 | RPS12 40S ribosomal protein S12 | 4 |
| 137 | 46.5 | IPI00917605.1 | CYCS Uncharacterized protein | 4 |
| 138 | 4.6 | IPI00409593.2 | LMO7 Isoform 4 of LIM domain only protein 7 | 3 |
| 139 | 25.9 | IPI00025512.2 | HSPB1 Heat shock protein beta-1 | 3 |
| 140 | 26.9 | IPI00956313.1 | KRT6C 58 kDa protein | 9 |
| 141 | 28.9 | IPI00012011.6 | CFL1 Cofilin-1 | 3 |
| 142 | 62.7 | IPI00017448.1 | RPS21; LOC100291837 40S ribosomal protein S21 | 3 |
| 143 | 16.4 | IPI00873201.1 | PSAP Isoform Sap-mu-6 of proactivator polypeptide | 3 |
| 144 | 7.3 | IPI00029717.1 | FGA Isoform 2 of fibrinogen alpha chain | 4 |
| 145 | 33.3 | IPI00910779.1 | YWHAG FLJ52141, highly similar to 14-3-3 protein | 7 |
| | | | gamma | |
| 146 | 18.1 | IPI00554788.5 | KRT18 Keratin, type I cytoskeletal 18 | 6 |
| 147 | 3.1 | IPI00298281.4 | LAMC1 Laminin subunit gamma-1 | 3 |
| 148 | 64.6 | IPI00939362.1 | S100A9 Uncharacterized protein | 4 |
| 149 | 24.4 | IPI00178440.3 | SNORA41; EEF1B2 Elongation factor 1-beta | 4 |
| 150 | 35.6 | IPI00006034.1 | CRIP2 Cysteine-rich protein 2 | 3 |
| 151 | 6.7 | IPI00789435.2 | EEF1D 71 kDa protein | 4 |
| 152 | 9.8 | IPI00298497.3 | FGB Fibrinogen beta chain | 4 |
| 153 | 17.4 | IPI00927101.1 | RPSAP15; SNORA6; RPSA; SNORA62 Uncharacterized | 3 |
| | | | protein | |
| 154 | 36.7 | IPI00829947.2 | IGKV1-5 Ig kappa chain V-II region GM607 | 3 |
| 155 | 4.6 | IPI00744889.2 | CDH1 E-cadherin | 3 |
| 156 | 24.2 | IPI00024933.3 | RPL12 Isoform 1 of 60S ribosomal protein L12 | 3 |
| 157 | 5.4 | IPI00293464.5 | DDB1; LOC100290337 DNA damage-binding protein 1 | 4 |
| 158 | 11.5 | PI00926319.1 | DLD FLJ56112, highly similar to dihydrolipoyl | 3 |
| | | | dehydrogenase, mitochondrial | |
| 159 | 24.8 | IPI00219806.7 | S100A7 Protein S100-A7 | 3 |
| 160 | 9.1 | IPI00232492.4 | TRIM29 Isoform Beta of Tripartite motif-containing | 4 |
| | | | protein 29 | |
| 161 | 66.7 | IPI00008527.3 | RPLP1 60S acidic ribosomal protein P1 | 4 |
| 162 | 16.3 | IPI00744692.1 | TALDO1 Transaldolase | 3 |
| 163 | 21.4 | IPI00924819.2 | MANF mesencephalic astrocyte-derived neurotrophic | 3 |
| | | | factor | |
| 164 | 38.7 | IPI00007047.1 | S100A8 Protein S100-A8 | 3 |
| 165 | 32.4 | IPI00019038.1 | LYZ Lysozyme C | 3 |
| 166 | 19.8 | IPI00940656.1 | ANP32A; LOC723972 28 kDa protein | 3 |
| 167 | 12.3 | IPI00910487.1 | SERPINH1 cDNA FLJ52569, highly similar to collagen- | 3 |
| | | | binding protein 2 | |
| 168 | 16.9 | IPI00024911.1 | ERP29 Endoplasmic reticulum resident protein 29 | 3 |
| 169 | 17.4 | IPI00304962.4 | COL1A2 Collagen alpha-2(I) chain | 9 |
| 170 | 9.7 | IPI00025366.4 | CS Citrate synthase, mitochondrial | 3 |
| 171 | 23.3 | IPI00022392.1 | C1QA Complement C1q subcomponent subunit A | 4 |
| 172 | 31.4 | IPI00216691.5 | PFN1 Profilin-1 | 3 |
| 173 | 34.9 | IPI00642816.2 | SRP9; SRP9L1 Signal recognition particle 9 kDa protein | 3 |
| 174 | 23.7 | IPI00007797.3 | FABP5 Fatty acid-binding protein, epidermal | 3 |
| 175 | 7.5 | IPI00917575.2 | HSPD1 cDNA FLJ51046, highly similar to 60 kDa heat | 3 |
| | | | shock protein, mitochondrial | |
| 176 | 5.5 | IPI00028931.2 | DSG2 Desmoglein-2 | 3 |
| 177 | 46.6 | IPI00017963.1 | SNRPD2 Small nuclear ribonucleoprotein Sm D2 | 3 |
| 178 | 16 | IPI00465084.6 | DES Desmin | 6 |
| 179 | 12.7 | IPI00792011.1 | CAPS Calcyphosin | 3 |
| 180 | 28.4 | IPI00216472.1 | CLTB Isoform non-brain of clathrin light chain B | 2 |
| 181 | 21.2 | IPI00217468.3 | HIST1H1B Histone H1.5 | 3 |
| 182 | 7.1 | IPI00022895.7 | A1BG Alpha-1B-glycoprotein | 2 |
| 183 | 9.6 | IPI00908881.2 | GPI Glucose-6-phosphate isomerase | 2 |
| 184 | 3 | IPI00001869.3 | PAPPA Pappalysin-1 | 2 |
| 185 | 19.1 | IPI00470498.1 | SERBP1 Isoform 3 of Plasminogen activator inhibitor 1 | 2 |
| | | | RNA-binding protein | |
| 186 | 5.5 | IPI00376403.2 | SPINT1 Isoform 1 of Kunitz-type protease inhibitor 1 | 2 |
| 187 | 15.6 | IPI00306959.11 | KRT7 Keratin, type II cytoskeletal 7 | 6 |
| 188 | 2.6 | IPI00420096.4 | PLEC Isoform 8 of plectin | 2 |
| 189 | 34.2 | IPI00418153.1 | IGHG3 Putative uncharacterized protein | 26 |
| | | | DKFZp686I15212 | |
| 190 | 31.7 | IPI00646748.1 | TPM2 Tropomyosin 2 | 10 |
| 191 | 14.3 | IPI00004550.5 | KRT24 Keratin, type I cytoskeletal 24 | 5 |
| 192 | 13.1 | IPI00942981.1 | DDRGKI 36 kDa protein | 2 |
| 193 | 5.5 | IPI00941899.1 | PKM2 66 kDa protein | 2 |
| 194 | 13.9 | IPI00645948.2 | HMGB1 High-mobility group box 1 | 2 |
| 195 | 23.5 | IPI00328840.9 | THOC4 THO complex subunit 4 | 2 |
| 196 | 12.3 | IPI00215911.3 | APEX1 DNA-(apurinic or apyrimidinic site) lyase | 2 |
| 197 | 8.2 | IPI00011107.2 | IDH2 Isocitrate dehydrogenase [NADP], mitochondrial | 2 |
| 198 | 1.8 | IPI00008868.3 | MAP1B Microtubule-associated protein 1B | 2 |
| 199 | 16.7 | IPI00967216.1 | HNRNPH1 Uncharacterized protein | 2 |
| 200 | 3 | IPI00956122.2 | CD44 Isoform 5 of CD44 antigen | 2 |
| 201 | 7.8 | IPI00916285.1 | MUC1 MUC1 isoform Z-LSP | 2 |
| 202 | 21.9 | IPI00908746.1 | PEBP1 cDNA FLJ51535, highly similar to | 2 |
| | | | phosphatidylethanolamine-binding protein 1 | |
| 203 | 3.3 | IPI00883857.2 | HNRNPU Isoform Long of Heterogeneous nuclear | 2 |
| | | | ribonucleoprotein U | |
| 204 | 19.7 | IPI00830130.1 | IGHV1-2 Rheumatoid factor RF-ET7 | 5 |
| 205 | 10 | IPI00828004.1 | COL18A1 Multi-functional protein MFP | 2 |
| 206 | 9.9 | IPI00643788.1 | LYPD3 Putative uncharacterized protein | 2 |
| | | | DKFZp686D0114 | |
| 207 | 12.7 | IPI00642213.1 | RALY RNA binding protein, auto-antigenic | 2 |
| 208 | 11.7 | IPI00552913.4 | STX7 Isoform 2 of syntaxin-7 | 2 |
| 209 | 11.2 | IPI00514330.5 | HDGF hepatoma-derived growth factor isoform c | 2 |
| 210 | 20.2 | IPI00302850.4 | SNRPD1 Small nuclear ribonucleoprotein Sm D1 | 2 |
| 211 | 10.4 | IPI00291922.2 | PSMA5 Proteasome subunit alpha type-5 | 2 |
| 212 | 9.8 | IPI00216493.1 | HNRNPH3 Isoform 3 of heterogeneous nuclear | 2 |
| | | | ribonucleoprotein H3 | |
| 213 | 8.2 | IPI00101037.3 | RCN3 Reticulocalbin-3 | 2 |
| 214 | 25 | IPI00068430.1 | SNRPEL1 Putative small nuclear ribonucleoprotein | 2 |
| | | | polypeptide E-like protein 1 | |
| 215 | 6 | IPI00029568.2 | PTX3 Pentraxin-related protein PTX3 | 2 |
| 216 | 13.7 | IPI00024920.1 | ATP5D ATP synthase subunit delta, mitochondrial | 2 |
| 217 | 8 | IPI00021840.1 | RPS6 40S ribosomal protein S6 | 2 |
| 218 | 45.5 | IPI00970878.1 | UBC 11 kDa protein | 3 |
| 219 | 23.3 | IPI00027463.1 | S100A6 Protein S100-A6 | 3 |
| 220 | 12.7 | IPI00946754.1 | PRSS1 Protease serine 1 | 6 |
| 221 | 9.1 | IPI00917753.1 | SET SET nuclear oncogene | 2 |
| 222 | 7 | IPI00328715.4 | MTDH Protein LYRIC | 2 |
| 223 | 2 | IPI00853454.1 | LAMB1 200 kDa protein | 2 |
| 224 | 5.9 | IPI00414335.2 | ILF3 Isoform 3 of interleukin enhancer-binding factor 3 | 2 |
| 225 | 33.3 | IPI00719622.1 | RPS28 40S ribosomal protein S28 | 3 |
| 226 | 4.6 | IPI00023673.1 | LGALS3BP Galectin-3-binding protein | 2 |
| 227 | 12.1 | IPI00024175.3 | PSMA7 Isoform 1 of proteasome subunit alpha type-7 | 2 |
| 228 | 11.7 | IPI00956475.1 | SEBOX cDNA FLJ51266, highly similar to vitronectin | 2 |
| 229 | 10.9 | IPI00791083.1 | PSMA4 20 kDa protein | 2 |
| 230 | 18.7 | IPI00555698.1 | CSDA CSDA protein variant (Fragment) | 2 |
| 231 | 8.5 | IPI00024157.1 | FKBP3 Peptidyl-prolyl cis-trans isomerase FKBP3 | 2 |
| 232 | 10.8 | IPI00550239.4 | H1F0 Histone H1.0 | 2 |
| 233 | 6.1 | IPI00018953.1 | DPP4 Dipeptidyl peptidase 4 | 2 |
| 234 | 18.6 | IPI00927864.2 | MDH2 Malate dehydrogenase | 1 |
| 235 | 19.4 | IPI00007702.1 | HSPA2 Heat shock-related 70 kDa protein 2 | 12 |
| 236 | 21 | IPI00883722.1 | LOC100293069; CFHR1 complement factor H-related 1 | 5 |
| 237 | 6.1 | IPI00954954.1 | CLU clusterin isoform 3 | 1 |
| 238 | 21.5 | IPI00375676.11 | FTL Ferritin | 1 |
| 239 | 15 | IPI00026199.2 | GPX3 Glutathione peroxidase 3 | 1 |
| 240 | 38.1 | IPI00887169.2 | IGLV1-47 Putative uncharacterized protein | 8 |
| 241 | 14.2 | IPI00015842.1 | RCN1 Reticulocalbin-1 | 2 |
| 242 | 0.8 | IPI00020557.1 | LRP1 Prolow-density lipoprotein receptor-related protein 1 | 1 |
| 243 | 10.9 | IPI00879750.1 | SNRPD3 19 kDa protein | 1 |
| 244 | 23.5 | IPI00549330.5 | IGKV3D-15 Myosin-reactive immunoglobulin light chain | 1 |
| | | | variable region | |
| 245 | 6.7 | IPI00872684.1 | EZR 69 kDa protein | 1 |
| 246 | 16.5 | IPI00795937.1 | CD9 15 kDa protein | 1 |
| 247 | 42.7 | IPI00922213.2 | FN1 cDNA FLJ53292, highly similar to fibronectin 1 | 29 |
| | | | (FN1) | |
| 248 | 50.3 | IPI00784817.1 | IGHG1; LOC100290146; IGHV4-31 anti-RhD monoclonal | 42 |
| | | | T125 gamma1 heavy chain | |
| 249 | 49.2 | IPI00399007.7 | IGHG2 Putative uncharacterized protein | 33 |
| | | | DKFZp686I04196 | |
| 250 | 36.5 | IPI00478672.5 | SCEL Isoform 2 of Sciellin | 17 |
| 251 | 56.6 | IPI00302047.3 | CAST cDNA FLJ77737, highly similar to calpastatin | 13 |
| | | | (CAST) | |
| 252 | 52 | IPI00894498.1 | ACTB Beta actin variant (fragment) | 13 |
| 253 | 48.6 | IPI00045396.2 | CALU Calumenin isoform 4 | 10 |
| 254 | 48.7 | IPI00969456.1 | IGKV1-39; IGKV1D-16; IGKV2-40; LOC100291464; IGKC | 19 |
| | | | Putative uncharacterized protein | |
| 255 | 49.7 | IPI00473011.3 | HBD Hemoglobin subunit delta | 8 |
| 256 | 64 | IPI00939160.2 | HBG2 G-gamma globin Paulinia variant | 7 |
| 257 | 12.4 | IPI00796776.1 | KRT5 cDNA FLJ54081, highly similar to Keratin, type II | 4 |
| | | | cytoskeletal 5 | |
| 258 | 8.5 | IPI00966854.1 | HNRNPAB Uncharacterized protein | 2 |
| 259 | 6.6 | IPI00883896.1 | LIMA1 LIM domain and actin-binding protein 1 isoform a | 1 |
| 260 | 1.5 | IPI00946286.1 | COL6A3 collagen alpha-3(VI) chain isoform 4 precursor | 1 |
| 261 | 23.7 | IPI00644668.2 | FHL1 Isoforrn 3 of Four and a half LIM domains protein 1 | 1 |
| 262 | 1.8 | IPI00005614.6 | SPTBN1 Isoform Long of spectrin beta chain, brain 1 | 1 |
| 263 | 5.7 | IPI00967527.1 | HEXB ENC-1AS | 1 |
| 264 | 3.8 | IPI00966603.1 | PDLIM5 Uncharacterized protein | 1 |
| 265 | 1.6 | IPI00956342.1 | MRC1L1 Mannose receptor, C type 1-like 1 | 1 |
| 266 | 9.2 | IPI00927255.1 | LANCL1 cDNA FLJ51489, highly similar to LanC-like | 1 |
| | | | protein 1 | |
| 267 | 10.5 | IPI00926019.1 | IGFBP1 Uncharacterized protein | 1 |
| 268 | 23.1 | IPI00871480.1 | UBL3 Uncharacterized protein | 1 |
| 269 | 4.9 | IPI00790831.1 | LMNB1 LMNB1 protein | 1 |
| 270 | 10.6 | IPI00645201.1 | SNORD55; RPS8 40S ribosomal protein S8 | 1 |
| 271 | 1.8 | IPI00643731.2 | COL17A1 Isoform 2 of collagen alpha-1(XVII) chain | 1 |
| 272 | 16.1 | IPI00642739.1 | TIMP1 TIMP metallopeptidase inhibitor 1 | 1 |
| 273 | 20.2 | IPI00455757.2 | RPL35 Similar to 60S ribosomal protein L35 | 1 |
| 274 | 4.3 | IPI00296141.4 | DPP7 Dipeptidyl peptidase 2 | 1 |
| 275 | 4.9 | IPI00216613.1 | SFPQ Isoform Short of Splicing factor, proline- and | 1 |
| | | | glutamine-rich | |
| 276 | 10.2 | IPI00152409.1 | AGR3 Anterior gradient protein 3 homolog | 1 |
| 277 | 9.2 | IPI00002745.1 | CTSZ Cathepsin Z | 1 |
| 278 | 9.4 | IPI00000494.6 | SNORD21; RPL5 60S ribosomal protein L5 | 1 |
| 279 | 5.1 | IPI00967483.1 | POLR2J2 30 kDa protein | 1 |
| 280 | 2.7 | IPI00953689.1 | AHSG Alpha-2-HS-glycoprotein | 1 |
| 281 | 14 | IPI00952829.1 | HMGA1 Uncharacterized protein | 1 |
| 282 | 8.7 | IPI00946221.1 | RPL24 Uncharacterized protein | 1 |
| 283 | 10 | IPI00943265.1 | IGKV4-1 Similar to Ig kappa chain V-IV region precursor | 1 |
| 284 | 3.8 | IPI00942944.1 | DEK protein DEK isoform 2 | 1 |
| 285 | 15.1 | IPI00925251.1 | SNRPG Uncharacterized protein | 1 |
| 286 | 2.1 | IPI00924935.1 | TFRC cDNA FLJ57106, highly similar to transferrin | 1 |
| | | | receptor protein 1 | |
| 287 | 3.9 | IPI00909623.1 | CNN3 cDNA FLJ53072, highly similar to calponin-3 | 1 |
| 288 | 7.3 | IPI00909586.1 | PAFAH1B1 cDNA FLJ52123, highly similar to platelet- | 1 |
| | | | activating factor acetylhydrolase IB alpha subunit | |
| 289 | 8.5 | IPI00909492.1 | SUMO3 cDNA FLJ57440, moderately similar to Small | 1 |
| | | | ubiquitin-related modifier 3 | |
| 290 | 7 | IPI00884926.1 | ORM1 alpha-1-acid glycoprotein 1 precursor | 1 |
| 291 | 8.5 | IPI00878669.2 | CBX1 Uncharacterized protein | 1 |
| 292 | 9.4 | IPI00798267.1 | CA1 14 kDa protein | 1 |
| 293 | 1.7 | IPI00785113.1 | LRRFIP1 Isoform 1 of Leucine-rich repeat flightless- | 1 |
| | | | interacting protein 1 | |
| 294 | 6.2 | IPI00465361.4 | RPL13 60S ribosomal protein L13 | 1 |
| 295 | 8.8 | IPI00443909.1 | CNPY2 Isoform 1 of protein canopy homolog 2 | 1 |
| 296 | 4.1 | IPI00329538.3 | PRSS8 Prostasin | 1 |
| 297 | 7.9 | IPI00298547.3 | PARK7 Protein DJ-1 | 1 |
| 298 | 5.7 | IPI00056334.5 | PRKCDBP Protein kinase C delta-binding protein | 1 |
| 299 | 7.4 | IPI00030843.1 | CLDN9 Claudin-9 | 1 |
| 300 | 3.7 | IPI00025019.3 | PSMB1 Proteasome subunit beta type-1 | 1 |
| 301 | 8.9 | IPI00022446.1 | PF4 Platelet factor 4 | 1 |
| 302 | 2.7 | IPI00019580.1 | PLG Plasminogen | 1 |
| 303 | 9.3 | IPI00004845.4 | NIPSNAP3A Protein NipSnap homolog 3A | 1 |
| 304 | 12.8 | IPI00000761.3 | LY6G6C Lymphocyte antigen 6 complex locus protein | 1 |
| | | | G6c | |
| 305 | 8 | IPI00871889.1 | PSMA1 Proteasome subunit alpha type | 1 |
| 306 | 2.6 | IPI00019988.1 | SGSH N-sulphoglucosamine sulphohydrolase | 1 |
| 307 | 14.9 | IPI00419249.5 | PSMA3 Isoform 1 of proteasome subunit alpha type-3 | 2 |
| 308 | 17.2 | IPI00940960.1 | NPC2 Epididymal secretory protein E1 | 1 |
| 309 | 8.5 | IPI00002535.2 | FKBP2 Peptidyl-prolyl cis-trans isomerase FKBP2 | 1 |
| 310 | 13.6 | IPI00012750.3 | RPS25 40S ribosomal protein S25 | 1 |
| 311 | 3.6 | IPI00749469.5 | PPA2 25 kDa protein | 1 |
| 312 | 1.8 | IPI00796316.4 | GSN cDNA FLJ53327, highly similar to gelsolin | 1 |
| 313 | 10.8 | IPI00550020.3 | PTMS Parathymosin | 1 |
| 314 | 9.3 | IPI00026328.3 | TXNDC12 Thioredoxin domain-containing protein 12 | 2 |
| 315 | 8.2 | IPI00794630.1 | CSRP2 14 kDa protein | 1 |
| 316 | 20.5 | IPI00009792.1 | IGHV1OR15-1 Ig heavy chain V-I region V35 | 4 |
| 317 | 18.5 | IPI00645510.1 | UFM1 Ubiquitin-fold modifier 1 | 1 |
| 318 | 8.8 | IPI00796198.1 | PSMB6 12 kDa protein | 1 |
| 319 | 24 | IPI00217466.3 | HIST1H1D Histone H1.3 | 7 |
| 320 | 2.5 | IPI00216057.6 | SORD Sorbitol dehydrogenase | 1 |
| 321 | 1.6 | IPI00375927.3 | WDR72 WD repeat-containing protein 72 | 1 |
| 322 | 1.1 | IPI00167953.3 | KBTBD3 kelch repeat and BTB domain-containing | 1 |
| | | | protein 3 | |
| 323 | 6.1 | IPI00647366.1 | CRYZ quinone oxidoreductase isoform b | 1 |
| 324 | 3.1 | IPI00939560.1 | TXNDC5 thioredoxin domain-containing protein 5 | 1 |
| | | | isoform 3 | |
| 325 | 2.2 | IPI00908404.1 | GNS cDNA FLJ51188, highly similar to N- | 1 |
| | | | acetylglucosamine-6-sulfatase | |
| 326 | 0.8 | IPI00607892.1 | ARHGAP33 Isoform 3 of Rho GTPase-activating protein | 1 |
| | | | 33 | |
| 327 | 27.6 | IPI00016179.1 | S100A13 Protein S100-A13 | 1 |
| 328 | 4.1 | IPI00221296.1 | PDLIM4 Isoform 2 of PDZ and LIM domain protein 4 | 1 |
| 329 | 6.1 | IPI00926261.1 | IGFBP3 Uncharacterized protein | 1 |
| 330 | 35.8 | IPI00893178.1 | IGLC3; IGLC1; IGLV1-44; IGLL5 23 kDa protein | 7 |
| 331 | 7.2 | IPI00295542.5 | NUCB1 Nucleobindin-1 | 1 |
| 332 | 12.9 | IPI00552768.1 | TXN Thioredoxin, isoform CRA_b | 1 |
| 333 | 1.3 | IPI00739940.4 | FRYL Isoform 1 of Protein furry homolog-like | 1 |
| 334 | 9.4 | IPI00011302.1 | CD59 CD59 glycoprotein | 0 |
| 335 | 0.6 | IPI00064162.4 | VCPIP1 Deubiquitinating protein VCIP135 | 0 |

TABLE 2
Significant pathways potentially affected by LAE (ASE) proteins using
MetaCore analysis

| | Significant Pathways | P values | LAE (ASE) proteins |
|---|---|---|---|
| 1 | Cytoskeleton remodeling: | 3.38 × 10⁻¹⁷ | ACTB, DSP, KRT-1, 2, 5, 6C, |
| | Keratin filaments | | 8, 14, 18, 19, LAMB1, LAMC1, |
| | | | MAP1B, PLEC, VIM, YWHAZ |
| 2 | Blood coagulation | 3.52 × 10⁻⁸ | A2M, A2ML1, CP, F13A1, |
| | | | FGA, FGB, PLG, SERBP1, |
| | | | SERPINA1 |
| 3 | Immune response: | 3.52 × 10⁻⁸ | C3, CD59, CFH, CFHR1, |
| | Alternative complement | | CLU, ILF3 |
| | pathway | | |
| 4 | Cell adhesion: ECM | 3.89 × 10⁻⁸ | CD44, COL1A1, COL1A2, |
| | remodelling | | COL3A1, EZR, FGB, FN1, |
| | | | LAMB1, LAMC1, LUM, NID1, |
| | | | OGN, PLG, SERBP1, TIMP1, |
| | | | VCAN |
| 5 | Immune response: Lectin- | 2.90 × 10⁻⁷ | C1QA, C1QC, C3, CD59, |
| | induced complement | | CLU, SERPING1 |
| | pathway | | |
| 6 | Cell adhesion: Cell-matrix | 4.49 × 10⁻⁷ | CD44, CP, FGB, ITIH1, ITIH2, |
| | glycoconjugates | | MUC1, LAMC1, LGALS3, |
| | | | LGALS3BP, LUM, OGN, PLG, |
| | | | TNC, VCAN |
| 7 | Immune response: | 4.94 × 10⁻⁷ | C1QA, C1QC, C3, CD59, |
| | Classical complement | | CLU, SERPING1 |
| | pathway | | |
| 8 | Protein folding and | 1.23 × 10⁻⁶ | AGT, CALR, FKBP3, PSMB1, |
| | maturation: Angiotensin | | SUMO3, UFM1, VCPIP1 |
| | system maturation | | |
| 9 | Transcription: Role of Akt in | 9.41 × 10⁻⁶ | ALDOA, APEX1, ENO1, |
| | hypoxia-induced HIF1 | | HSPA1B, HSPA2, PGK1, |
| | activation | | TFRC |
| 10 | Development: TGFβ- | 2.33 × 10⁻⁵ | ACTB, CALD1, CDH1, CFL1, |
| | signaling via RhoA, Pi3K, | | FN1, LTBP2, TGFBI, TPM1, |
| | ILK | | VIM |
| 11 | Glycolysis and | 3.34 × 10⁻⁵ | ALDOA, ENO1, GPI, MDH2, |
| | gluconeogenesis (short | | ORM1, PGK1, TPI1 |
| | map) | | |
| 12 | Cytoskeleton remodeling: | 9.33 × 10⁻⁵ | ACTB, DCTN2, DES, MAP1B, |
| | Neurofilaments | | PLEC, VIM |

### Immunofluorescence

### Hanging Drop Culture

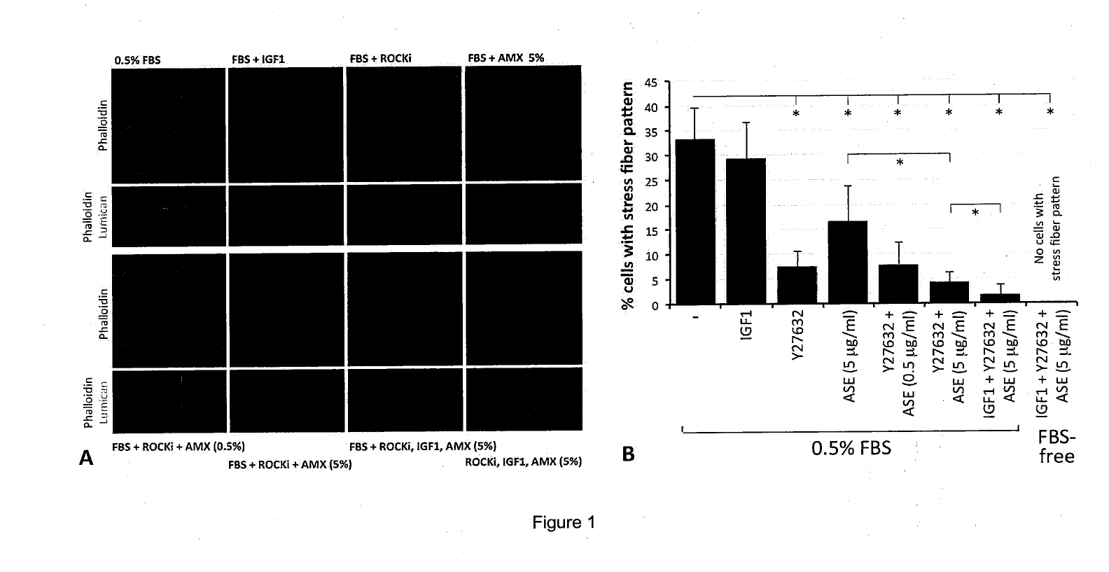

Human corneal stromal fragments were digested with collagenase I (0.1% in KBM) to single cell suspension and cultured in KBM with different supplements. The stress fiber reduction of CSKs cultured in KBM with different supplements is shown in FIG. 1. Fibroblast transformation is related to F-actin stress fiber induction. FIG. 1(A) shows that by phalloidin-AlexaFluor543 staining, primary human CSKs in KBM supplemented with 0.5% FBS and LAE (ASE), ROCKi Y27632 and IGF1 (termed as KBM+0.5% SERI) showed negligible cytoplasmic stress fibers (2-7% of total cells; FIG. 1(B)) Instead, the cells had predominant cortical F-actin alignment pattern. This was much lower than cells in serum (>33%, FIG. 1(B)). *P<0.05, One-way ANOVA with Dunn-Bonferroni correction. When put in serum culture (0.5% FBS), primary human CSKs appeared proliferative and displayed prominent cytoplasmic F-actin stress fibres by phalloidin staining (FIG. 1(A)). This was less detectable in cells cultured in KBM with ROCK inhibitor, Y27632 (10 μM) (FIG. 1(A)). All treated cells showed phalloidin reactivity but had different fibre pattern. Calculated from the total number of cells and cells with cortical F-actin only (non-stress cells), the percentage of cells with stress fibre pattern (stress index) was obtained. In 5 random fields, the stress index of CSK in KBM+0.5% FBS was 33.3±6.4% and it was significantly reduced to 7.6±2.9% when Y27632 was added (P<0.05, One-way ANOVA with Dunn-Bonferroni correction) (FIG. 1(B)). It was also observed that lumican was undetectable in serum-added culture but appeared in KBM+serum-free ERI (FIG. 1A).

Primary CSK maintained the typical dendritic morphology and expressed keratocyte-associated genes when they were cultured in AM stromal matrix even in the presence of serum (Espana et al., 2003). However, such culture method is not practically feasible, in particular for routine viewing of cells and subpassaging. Cultivation of primary CSKs with soluble LAE (ASE) was tested. After keeping in KBM+0.5% FBS supplemented with LAE (ASE) (5 μg protein/ml) for 7 days, CSK showed moderate cell proliferation and low stress index. There were 16.6±7.1% cells showing the polarity pattern of F-actin fibers. Co-treatment with Y27632 (10 μM) further reduced the stress index. It was dose-dependent to LAE (ASE) (7.9±4.3% for LAE (ASE) at 0.5 μg protein/ml and 4.1±2.1% for LAE (ASE) at 5 μg protein/ml) (FIG. 1(B)). IGF1 was shown to improve collagen secretion of rabbit KC (Lakshman et al., 2012). Prominent stress F-actin fibre was seen in IGF1-treated cells (29.3±7.3% cells with 0.5% FBS) (FIG. 1(B)). When the cells were cultured with Y27632 (10 μg/ml), LAE (ASE) (5 μg protein/ml) and IGF1 (10 ng/ml), there was low to negligible stress index (1.6±2.2% in 0.5% FBS and 0% in serum-free condition). The keratocyte gene, lumican, was negligibly expressed in cells cultured in serum, irrespective to the combination of supplements added (FIG. 1A). It expressed in KBM+ERI-cultured cells but not in serum culture.

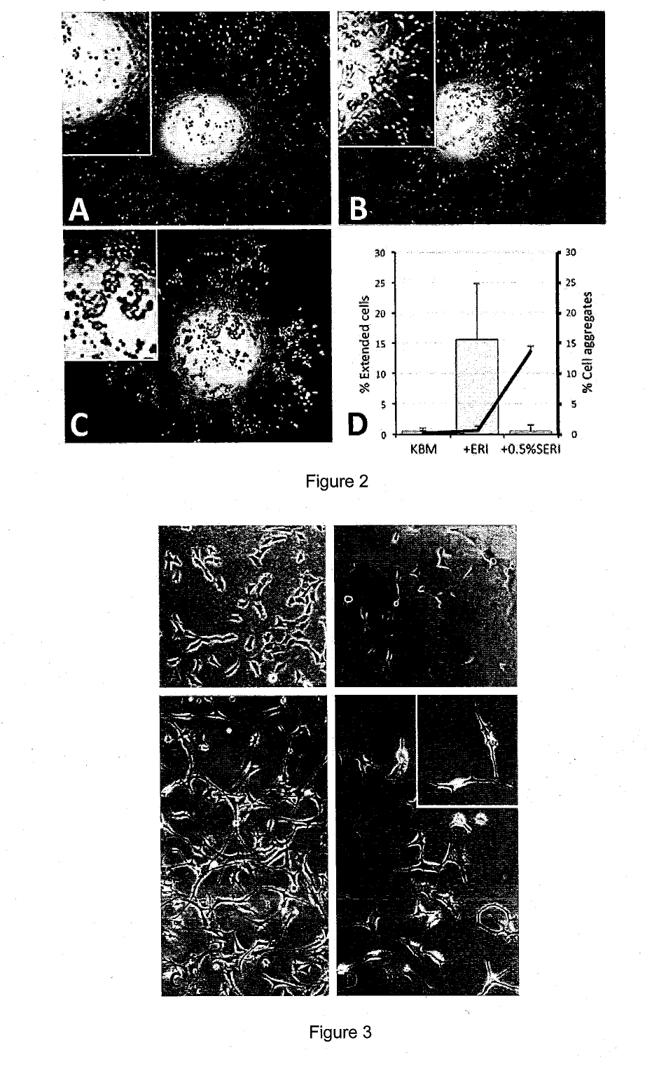

The culture of CSK in this KBM+ERI cocktail under attachment-free condition using hanging drop method was tested. Drops of cell suspension in different medium conditions were applied to the sterile surface, which was subsequently inverted to culture for 96 hours. Phase contrast images were taken at the center of each drop with focus on the meniscus (air-liquid interface). Cell quantification showed that 15.6±9.3% cells in KBM+ERI culture (FIG. 2(B)) had extended cell shape, significantly higher than those cells in KBM without ERI cocktail (serum-free: 0.5±0.6%, FIG. 2(A); 0.5% FBS: 0.5±0.8%, FIG. 2C)) (P<0.05, One-way ANOVA with Dunn-Bonferroni correction) (FIG. 2(D)). When cells were suspended in KBM+0.5% SERI (FIG. 2(C)), they formed aggregates. (13.6±0.9%), which were rarely observed in other conditions (serum-free KBM: 0.2±0.4%; KBM+0.5% FBS: 0.9±0.9% and KBM+ERI: 0.7±0.7%) (P<0.05, One-way ANOVA with Dunn-Bonferroni correction) (FIG. 2(D)).

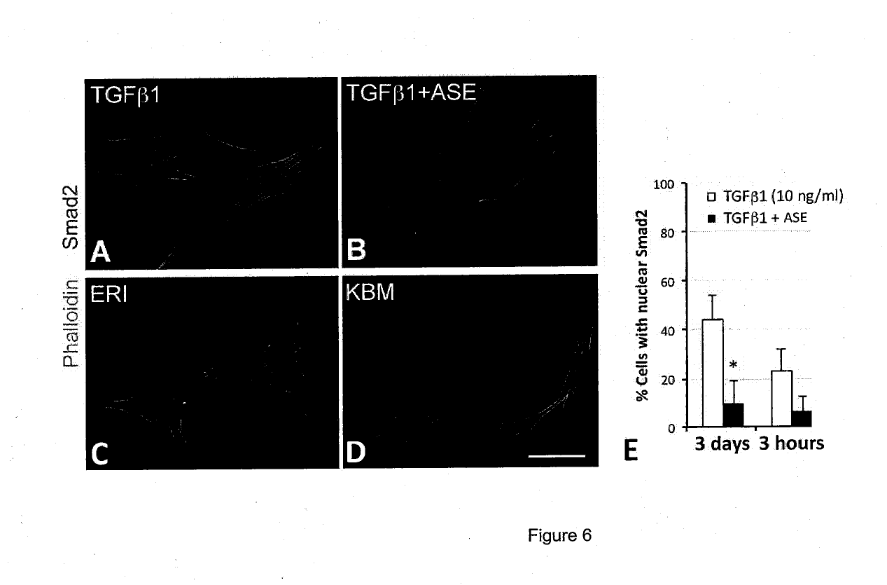

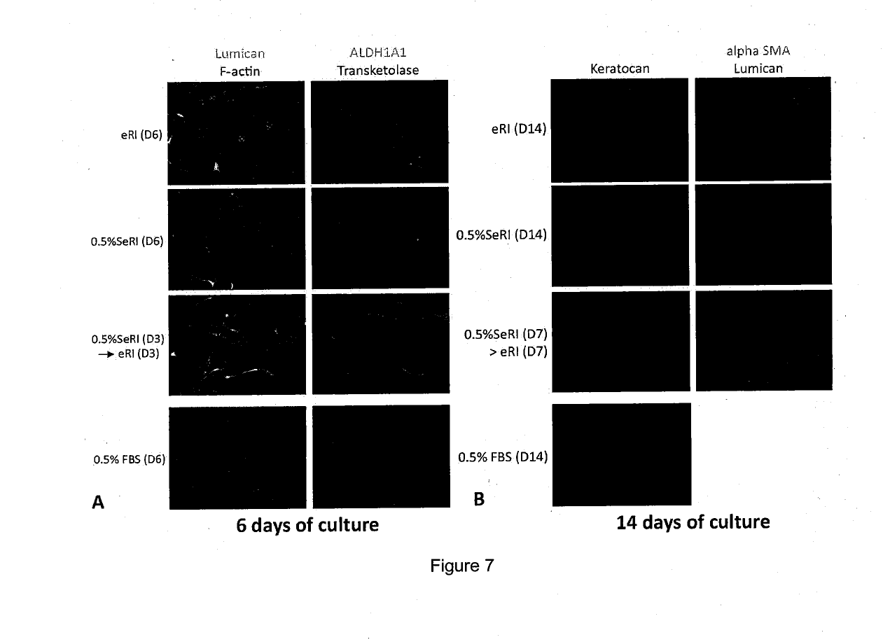

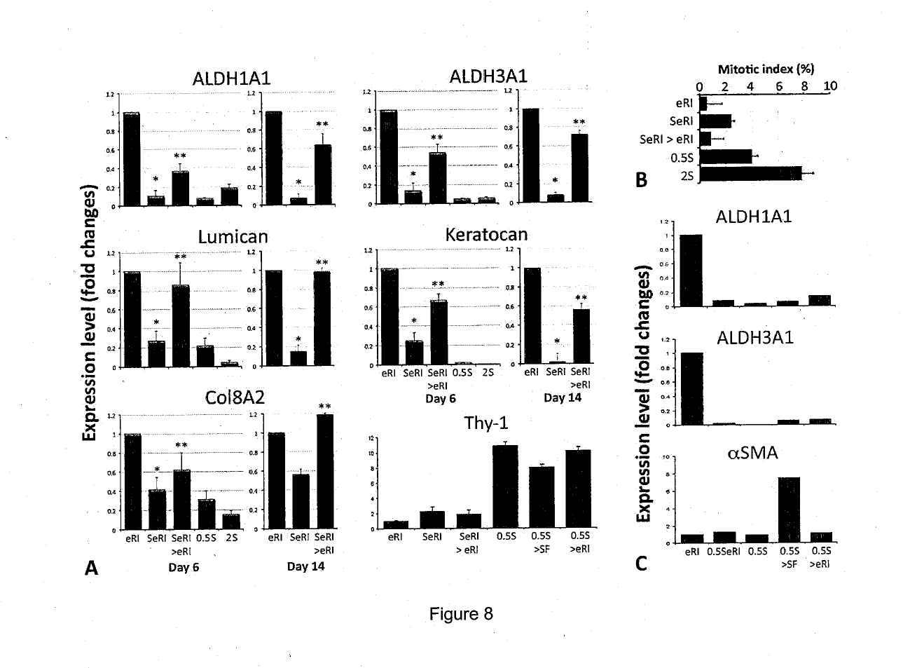

In culture on collagen I-coated surface, CSKs appeared as short slender shape in serum culture (either 2% or 0.5% FBS) (FIGS. 3(A) and 3(B)). Mitotic figures were frequently seen as the culture progressed. At day 6, there were 2.4±0.2% mitotic cells in 0.5% serum culture and only 0.6±1.1% mitotic cells in serum-free condition (FIG. 8B). At day 20, the mitotic index was 7.8±0.9% and 4±0.4% for cells in 2% and 0.5% FBS conditions, respectively (n=4). In KBM+0.5% SERI, human CSKs appeared stellate in shape with short processes interconnecting among cells (FIG. 3(C)). At day 3, the mitotic index was 2.4±0.2%. Typical CSKs were quiescent under serum-free condition. They had convoluted cell body and long and thin dendritic processes extending to neighboring cells forming cellular network (FIG. 3(D)). They strongly expressed lumican and ALDH1A1 (FIG. 7(A)) but were devoid of stress F-actin fiber pattern (FIG. 6(A)).

### ERI Inhibited Fibroblast-Mediated Collagen Gel Contraction

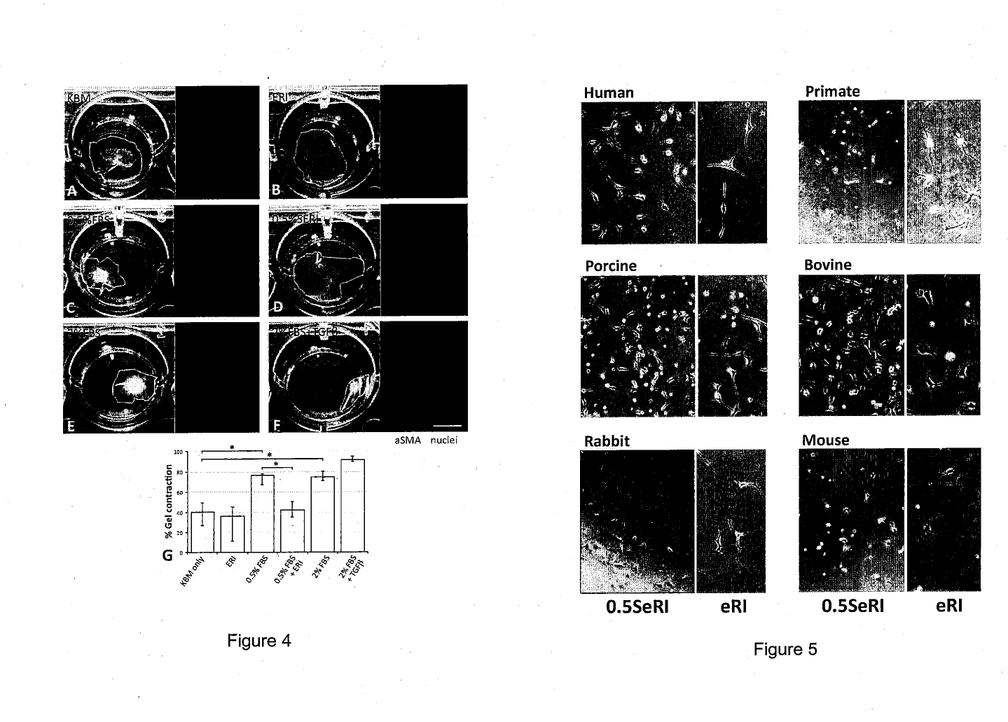

The functional contractile activity of fibroblasts derived from human CSKs (at passage 6) was studied by the collagen gel contraction assay. For cells in KBM only, the resultant gel contraction was 40.2% (median) (IQR: 27.4-50.1%) (FIG. 4(A)). It was increased to 76.1% and 74.4% for cells in KBM with 0.5% and 2% FBS, respectively (both had P<0.05, Mann-Whitney U test; compared to KBM only) (FIGS. 4(C), (E), (G)). When TGFβ1 (20 ng/ml) was added, the median percentage of gel contraction was further raised to 91.7% (IQR: 90.2-92.4%) (FIGS. 4(F), (G)). The application of ERI almost negated this change and the gel contraction was 36.5% (12.9-44.9%) for KBM+ERI and 42.1% (35.3-49.5%) for KBM+0.5% SERI, respectively. Both had no significant difference when compared to cells in KBM only. Comparing CSK cultures with KBM+0.5% FBS, there was significant reduction of gel contraction when ERI supplement was added (P=0.002, Mann-Whitney U test) (FIGS. 4B, D, G). Expression of cellular αSMA was observed in the contracted gels with 0.5% and 2% FBS added or not with TGβ1 (FIGS. 4(C), (E), (F)). Negligible staining was found in gels with KBM only, and KBM with ERI or KBM with 0.5% SERI (FIGS. 4(A), (B), (D)).

### ERI Culture of Animal Keratocytes

The cultivation of primary CSKs from different animal species (non-human primate, cow, pig, rabbit and mouse) using the standardized ERI cocktail (FIG. 5) was tested. Since all animal eyes were freshly collected and processed (less than 6 hours from death), the isolated keratocytes had higher viability and attachment efficiency than donor human keratocytes. When plated at 10⁴ cells per cm² on collagen I coated surface, the attachment efficiency at 48-hour interval was 30-40% for primate CSKs, 40% for rabbit CSKs, more than 50% for bovine and porcine CSKs. The viability of mouse CSKs was lower than expected (less than 10%) which could be due to the thinner and fragile stroma and cell damages during processing. All CSKs were moderately proliferative in KBM+0.5% SERI and maintained the typical dendritic morphology and established extensive intercellular contacts via cell processes, similar to the human CSK culture. No fibroblasts were seen in these cultures after 14 days.

### LAE (ASE) Suppressed TGFβ-Induced Nuclear Smad2/3 Localization

To examine if LAE (ASE) was effective in suppressing CSK transition to fibroblasts, TGFβ-mediated nuclear localization of Smad2/3 was studied. As shown in FIG. 6, human CSKs at passage 5 plated on collagen I-coated culture surface (10⁴ cells/cm²) were responsive differently to the addition of recombinant human TGFβ1 (10 ng/ml) and supplements. Nuclear Smad2/3 was detected in 12.5% cells without TGFβ1 but increased to 43.8% after 3 days of TGFβ challenge (22% when incubated for 3 hours) (FIG. 6(E)). When compared to TGFβ1-treated group, incubation with LAE (ASE) significantly suppressed nuclear Smad2/3 localization (P<0.05; one-way ANOVA with Dunn-Bonferroni correction).

### ERI Reverted Activated Keratocytes to Keratocytes

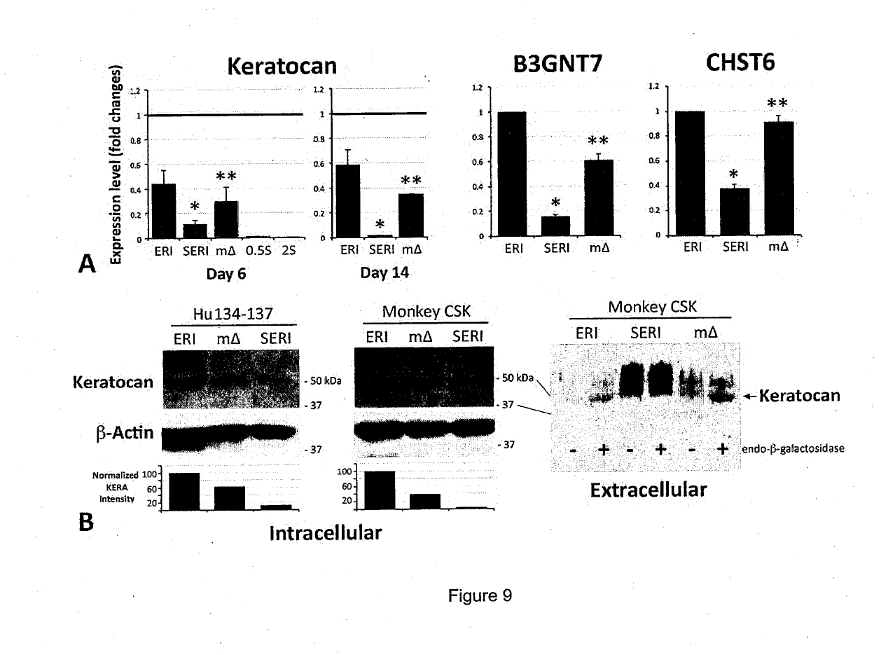

Human CSKs in KBM+0.5% SERI had moderate proliferation without fibroblast conversion. Instead, they became “activated keratocytes” with a characteristic suppression of keratoctye specific genes. By immunofluorescence, ALDH1A1, lumican and keratocan were down-regulated with human CSKs cultured in KBM+0.5% SERI for 6 and 14 days, respectively (row 2 in FIGS. 7(A) and 7(B)), when compared to KBM+ERI culture. This was corroborated by qPCR analysis, which additionally showed a reduced expression of ALDH3A1 and Col8A2 (FIG. 8(A)) and keratocan (FIG. 9(A)) Thy-1 expression was slightly induced in these activated keratocytes but not reaching the level exhibited by fibroblast cells in serum culture (FIG. 8(A)).

To investigate if these activated keratocytes could be reverted to keratocytes under KBM+ERI culture, the culture medium was switched from KBM+0.5% SERI to KBM+serum-free ERI at day 3 and 7, respectively. At the end of experiment, it was found that ALDH1A1, lumican and keratocan re-expressed under immunofluorescence (row 3 in FIGS. 7(A) and 7(B)). This was confirmed by qPCR study, which in addition showed regained expression of ALDH3A1 and Col8A2 (FIG. 8(A)) and keratocan (FIG. 9(A)). The efficiency of recovery for various keratocyte genes was calculated. They were 40% at day 6 and 64% at day 14 for ALDH1A1; 55% at day 6 and 72% at day 14 for ALDH3A1; 82% at day 6 and 98% at day 14 for lumican; 65% at day 6 and 56% at day 14 for keratocan as well as 60% at day 6 and 118% at day 14 for Col8A2. Simultaneously, Thy-1 expression was reduced (FIG. 8(A) and FIG. 9(A)). The cell morphology after medium switch appeared as typical keratocytes, similar as in KBM+serum-free ERI culture from the beginning (row 3 in FIGS. 7(A) and 7(B)).

To examine whether such ERI effect could be replaced by serum-free condition, cultured human CSKs were cultured in KBM+0.5% FBS for 3 days and subsequently switched to culture in serum-free basal medium or ERI-supplemented medium. In both cases, ALDH1A1 and ALDH3A1 did not regain their expression (FIG. 8(C)). Surprisingly, the expression of αSMA was elevated by 8 folds when cells were returned to serum-free basal medium, while it remained at low levels when switched back to KBM+ERI culture.

Keratocan Expression and Secretion in Expanded CSK after Media Switch

To demonstrate the functional phenotype of CSKs, keratocan protein expression in expanded CSKs at passage 5 under KBM+ERI culture or with media switch was studied. Keratocan migrating as a 50-kDa band in CSK lysates after digestion with endo-β-galactosidase was detected (FIG. 9B). Both human and monkey CSKs had stronger expression when cultured under KBM+ERI while faint detection in KBM+0.5% SERI for 14 days. When the media switch from KBM+0.5% SERI to KBM+ERI was performed at day 7, keratocan expression reappeared and was consistent with qPCR finding (FIG. 9(A)). Band densitometry assay showed that the keratocan protein expression in human CSKs under KBM+0.5% SERI was about 12% when compared to cells under KBM+ERI culture and the level was regained to 63% after media switch. Similarly, in monkey CSKs, the keratocan expression under KBM+0.5% SERI culture was 3.9% when compared to KBM+ERI culture and was retrieved to 39.5% after media switch (FIG. 9(B)). Because keratocan expression was strongly observed in the extracellular stromal matrix of in vivo corneas, conditioned media from monkey CSK cultures were also examined. The result showed that the digested samples of conditioned media from 5×10⁵ cells under KBM+ERI culture for 14 days and from the media switch condition (7 days in KBM+0.5% SERI followed by 7 days in KBM+ERI) had the 50-kDa protein band consistent with keratocan (FIG. 9(B)). No keratocan was detected in cell medium under KBM+0.5% SERI. Together with B3GNT7 and CHST6 expression (FIG. 9(A)), this indicated that ERI supplement maintained keratocan biosynthesis, expression and secretion in vitro.

### Frozen Storage and Retrieval of Viable “Activated Keratocytes”

“Activated keratocytes” expanded in KBM+0.5% SERI until 60% confluence were trypsinized and collected as cell suspension. After spinning, the cells were washed with sterile PBS and recovered as pellet. They were then suspended in KBM+0.5% SERI added with 0.5% dimethylsulfoxide (Sigma) at a concentration of 5×10⁵ cells/ml. The aliquots were immediately placed in the air phase of liquid nitrogen for 4 hours until frozen and were then immersed in liquid nitrogen for storage. After one and three weeks of frozen storage, the aliquots were retrieved from liquid nitrogen, warmed to 37° C. and diluted in KBM+0.5% SERI at a ratio of 1 ml:10 ml (vol/vol). The cell suspension was spun to recover cell pellet which was subsequently suspended in KBM+0.5% SERI, and plated at 10⁴ cells per cm² on collagen I-coated culture surface. At time of 60% confluence, mitotic index was monitored as 2.25±1.3% (n=3). This was insignificant different to primary human CSKs without any cell freezing and thawing procedures (2.4±0.2%) (P>0.05, paired Student's t-test).

### Expanded Human Keratocytes in Plastic Compressed Collagen

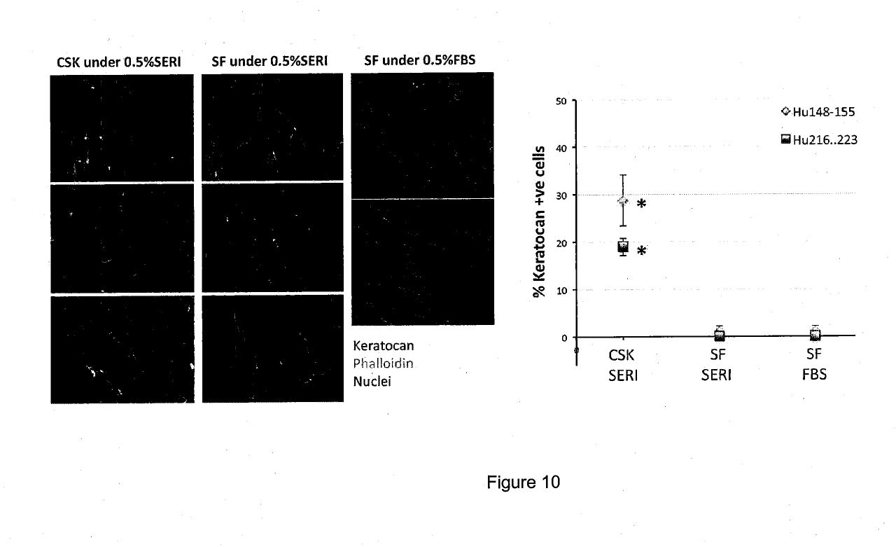

A study was carried out to examine if human CSKs expanded under KBM+ERI expressed proper keratocyte features when cells were cultured in plastic compressed collagen, which is a potential biological scaffold mimicking stromal architect. After 3 weeks in culture, cytoplasmic keratocan expression in human CSKs cultivated with KBM+0.5% SERI in compressed collagen matrix was observed (FIG. 10). However, it was not detectable in human stromal fibroblast (SF) (pre-expanded in KBM+serum only) cultured with KBM+0.5% SERI or KBM+0.5% FBS. Cell quantification showed that 28.8±5.4% (mean±SD) and 19.1±1.9% keratocan positive cells in two different human CSK cultures under KBM+0.5% SERI. These levels were significantly higher than SF cultures (close to 0%) (P<0.05, multiple comparison using Kruskal Wallis test and Dunn-Bonferroni correction).

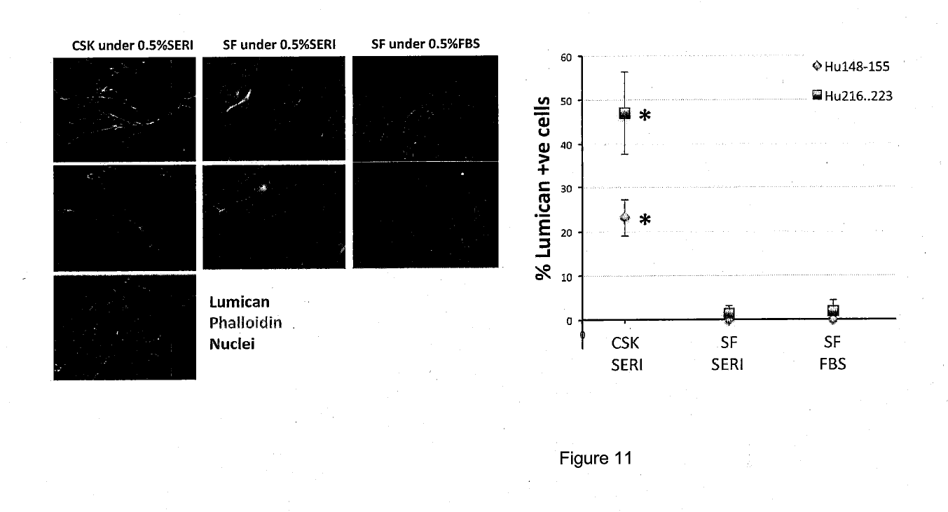

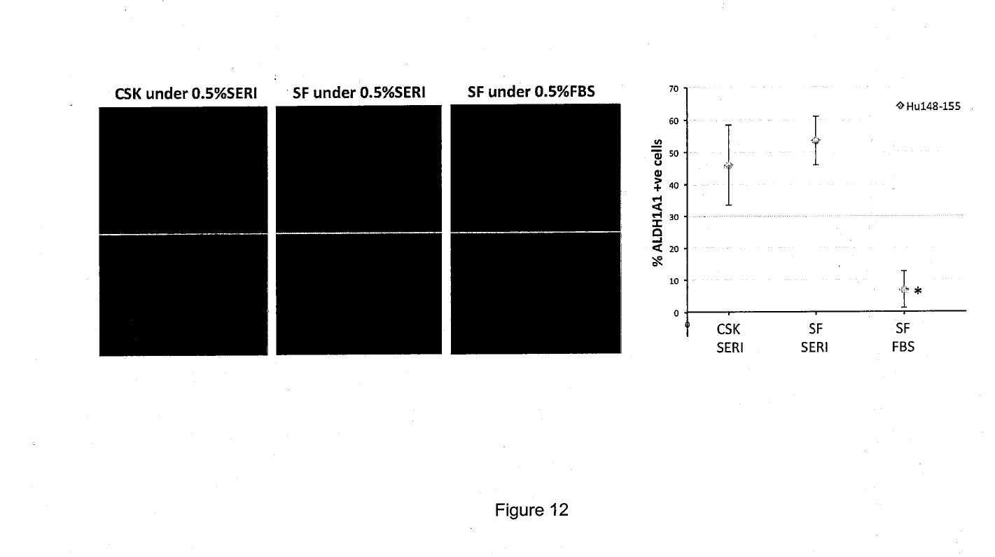

Similarly, cytoplasmic lumican was detected in human expanded CSKs cultivated with KBM+0.5% SERI in compressed collagen after 3 weeks but low to negligibly expressed in SF cultured with KBM+0.5% SERI or KBM+0.5% FBS (FIG. 11). Cell quantification illustrated 23.1±4.1% and 47.1±9.5% cells from two different human CSK cultures under 0.5% SERI expressing lumican. Again, these percentages were significantly higher than SF cultures (close to 0%) (P<0.05, multiple comparison using Kruskal Wallis test and Dunn-Bonferroni correction).

ALDH1A1 was also detected in expanded human CSKs under KBM+0.5% SERI (45.9±12.5% cells) after antigen retrieval by methanol treatment (FIG. 12). Similar expression was observed in 53.4±7.6% SF under KBM+0.5% SERI culture. However, SF cultured with KBM+0.5% FBS had significantly reduced ALDH1A1 positive cells (6.9±5.6%) (P<0.05, multiple comparison using Kruskal Wallis test and Dunn-Bonferroni correction).

### DISCUSSION

A novel culture protocol for ex vivo expansion of corneal stromal keratocytes without fibroblastic changes has been developed and is described herein. The method does not require the cells to be in contact with any composite. This avoids the presence of non-opaque composite which prevent easy viewing and monitoring of cells during culture.

In particular, this protocol employs an ERI cocktail as supplementation to the low serum culture of primary CSKs. The cells are moderately proliferative, display typical dendritic keratocyte morphology and have a transient loss of keratocyte-specific genes, but do not express any fibroblast-related genes. All these evidence indicate that the expanded cells are “activated keratocytes”. When these cells are returned to KBM+serum-free ERI condition, the keratocyte-specific gene suppression is retrieved. Such effect is not observed in cells not cultivated in KBM+ERI.

The expanded “activated keratocytes” can be stored frozen under liquid nitrogen for intermediate to extended periods of time and thawed to retrieve viable cells for continuous culture. This study identified for the first time the propagation of “activated keratocytes”, which could be reverted to genuine keratocytes for stromal tissue construction. This includes cell replacement therapy using intrastromal cell injection. Single cells are suspended in a medium (including normal saline, phosphate buffered saline and any types of isotonic buffer) from a density of 10⁴ to 10⁸ cells/ml and a volume of cell suspension is injected to the central or peripheral intrastromal site at different stromal depth levels by using a calibrated syringe equipped with a fine-pore needle (the pore size range is from 27G to 35G) controlled manually or electronic syringe pump or micro-injection device. Moreover, the cells can be first implanted to culture in a variety of biological and synthetic matrices, including decellularized human and animal corneal stroma tissue (full and partial thickness), decellularized human amniotic membrane, epithelial mucosa, collagen gel matrix (such as compressed collagen and hydrogel), fibrin gel, woven or non-woven silk biomaterials or bioscaffolds, polylactic acid based polymer membrane, fabricated polycaprolactone nanofibre scaffolds, electrospun polymeric mesh/matrix, polyurethane/gelatin composites and so on. Single cells at different density will be plated on or injected into the matrix and cultured for any period of time until transplantation. The cell/bioscaffold construct will then be surgically transplanted to the corneal stroma (including onlay and intrastromal pocket implantation) of recipient.

In summary, the CSK culture protocol has been refined by using the amnion stromal extract fractions together with cytokines and serum on collagen I-coated culture surface. This is basically a chemical type of reaction. The results showed the ex vivo expansion of activated keratocytes with correct dendritic morphology and negligible collagen gel contractibility. The cells expressed keratocyte-specific gene profile when returned to the serum-free condition. Culture of these cells in plastic compressed collagen further exhibited typical keratocyte gene expression and networking.

### REFERENCES

Chan A A, Hertsenberg A J, Funderburgh M L, Mann M M, Du Y, Davoli K A, Mich-Basso J D, Yang L, Funderburgh J L. Differentiation of human embryonic stem cells into cells with corneal keratocyte phenotype. PLoS One 8, e56831 (2013).
Du Y, Carlson E C, Funderburgh M L, Birk D E, Pearlman E, Guo N, Kao W W, Funderburgh J L. Stem cell therapy restores transparency to defective murine corneas. Stem Cells 27, 1635-1642 (2009).
Espana E M, He H, Kawakita T, Di Pascuale M A, Raju V K, Liu C Y, Tseng S C. Human keratocytes cultured on amniotic membrane stroma preserve morphology and express keratocan. Invest Ophthalmol Vis Sci 44, 5136-5141 (2003).
Funderburgh M L, Mann M M, Funderburgh J L. Keratocyte phenotype is enhanced in the absence of attachment to the substratum. Mol Vis 14, 308-317 (2008).
Kawakita T, Espana E M, He H, Hornia A, Yeh L K, Ouyang J, Liu C Y, Tseng S C. Keratocan expression of murine keratocytes is maintained on amniotic membrane by down-regulating transforming growth factor-beta signaling. J Blol Chem 280, 27085-27092 (2005).
Kawakita T, Espana E M, He H, Smiddy R, Parel J M, Liu C Y, Tseng S C. Preservation and expansion of the primate keratocyte phenotype by downregulating TGF-beta signaling in a low-calcium, serum-free medium. Invest Ophthalmol Vis Sci 47, 1918-1927 (2006).
Lakshman N, Petroll W M. Growth factor regulation of corneal keratocyte mechanical phenotypes in 3-D collagen matrices. Invest Ophthalmol Vis Sci 53, 1077-1086 (2012).
Lee S B, Li D Q, Tan D T, Meller D C, Tseng S C. Suppression of TGF-beta signaling in both normal conjunctival fibroblasts and pterygial body fibroblasts by amniotic membrane. Curr Eye Res 20, 325-334 (2000).
Levis H J, Peh G S, Toh K P, Poh R, Shortt A J, Drake R A, Mehta J S, Daniels J T. Plastic compressed collagen as a novel carrier for expanded human corneal endothelial cells for transplantation. PLoS One 7, e50993 (2012).
Li W, He H, Chen Y T, Hayashida Y, Tseng S C. Reversal of myofibroblasts by amniotic membrane stromal extract. J Cell Physiol 215, 657-664 (2008).
Pinnamaneni N, Funderburgh J L. Concise review: Stem cells in the corneal stroma. Stem Cells 30, 1059-1063 (2012).
Scott S G, Jun A S, Chakravarti S. Sphere formation from corneal keratocytes and phenotype specific markers. Exp Eye Res 93, 898-905 (2011).
Tseng S C, Li D Q, Ma X. Suppression of transforming growth factor-beta isoforms, TGF-beta receptor type I I, and myofibroblast differentiation in cultured human corneal and limbal fibroblasts by amniotic membrane matrix. J Cell Physiol 179, 325-335 (1999).
Wu J, Du Y, Watkins S C, Funderburgh J L, Wagner W R. The engineering of organized human corneal tissue through the spatial guidance of corneal stromal stem cells. Biomaterials 33, 1343-1352 (2012).
Yoshida S, Shimmura S, Shimazaki J, Shinozaki N, Tsubota K. Serum-free spheroid culture of mouse corneal keratocytes. Invest Ophthalmol Vis Sci 46, 1653-1658 (2005).

1. A method for culturing corneal stromal keratocytes, the method comprising:
(i) providing a population of corneal stromal keratocytes (CSKs) comprising at least one corneal stromal keratocyte (CSK);
(ii) contacting the population of CSKs with a culture medium A comprising a liquid amnion extract and serum; and
(iii) replacing the culture medium A with a culture medium B comprising a liquid amnion extract or a minimum essential medium.

2. The method according to claim 1, wherein the culture medium A and/or the culture medium B further comprises at least one Rho associated protein kinase inhibitor (ROCKi) and/or at least one insulin-like growth factor (IGF).

3. (canceled)

4. The method according to claim 1, wherein the culture medium B is free of serum or substantially free of serum.

5. The method according to claim 1, wherein the culture medium A comprises a minimum essential medium supplemented with serum and a liquid amnion extract and/or the culture medium B comprises a minimum essential medium supplemented with a liquid amnion extract.

6-11. (canceled)

12. The method according to claim 1, wherein the culture medium A comprises 0.1% to 10% serum.

13-14. (canceled)

15. The method according to claim 1, wherein the liquid amnion extract is derived from a mammal.

16. (canceled)

17. The method according to claim 2, wherein the ROCKi is selected from the group consisting of: (1R,4r)-4-((R)-1-aminoethyl)-N-(pyridin-4-yl)cyclohexanecarboxamide (Y-27632), 5-(1,4-diazepane-1-sulfonyl)isoquinoline (Fasudil), N-benzyl-2-(pyrimidin-4-ylamino)thiazole-4-carboxamide (Thiazovivin), N-(6-fluoro-1H-indazol-5-yl)-2-methyl-6-oxo-4-(4-(trifluoromethyl)phenyl)-1,4,5,6-tetrahydropyridine-3-carboxamide (GSK429286A), 1-(3-Hydroxybenzyl)-3-(4-(pyridin-4-yl)thiazol-2-yl)urea (RKI-1447 or ROCK inhibitor XIII), and a combination thereof.

18. The method according to claim 2, wherein the insulin-like growth factor comprises insulin-like growth factor 1 (IGF1) or insulin-like growth factor 2 (IGF2).

19. The method according to claim 1, wherein the method is carried out in the presence of collagen.

20-21. (canceled)

22. An isolated population of corneal stromal keratocytes (CSKs) obtainable/obtained by the method according to claim 1.

23. A culture medium B comprising a liquid amnion extract.

24. The culture medium B according to claim 23 further comprising at least one Rho associated protein kinase inhibitor (ROCKi) and/or at least one insulin-like growth factor (IGF).

25. (canceled)

26. The culture medium B according to claim 23, wherein the culture medium is free of serum or substantially free of serum.

27-29. (canceled)

30. A culture medium A comprising the culture medium B according to claim 23 and further comprising serum.

31-33. (canceled)

34. The culture medium B according to claim 23, wherein said culture medium B comprises a minimum essential medium supplemented with the liquid amnion extract.

35-39. (canceled)

40. The culture medium B according to claim 24, wherein the ROCKi is selected from the group consisting of: (1R,4r)-4-((R)-1-aminoethyl)-N-(pyridin-4-yl)cyclohexanecarboxamide (Y-27632), 5-(1,4-diazepane-1-sulfonyl)isoquinoline (Fasudil), N-benzyl-2-(pyrimidin-4-ylamino)thiazole-4-carboxamide (Thiazovivin), N-(6-fluoro-1H-indazol-5-yl)-2-methyl-6-oxo-4-(4-(trifluoromethyl)phenyl)-1,4,5,6-tetrahydropyridine-3-carboxamide (GSK429286A), 1-(3-Hydroxybenzyl)-3-(4-(pyridin-4-yl)thiazol-2-yl)urea (RKI-1447 or ROCK inhibitor XIII), and a combination thereof.

41. The culture medium B according to claim 24, wherein the insulin-like growth factor comprises insulin-like growth factor 1 (IGF1) or insulin-like growth factor 2 (IGF2).

42. The culture medium B according to claim 23, wherein said culture medium B further comprises collagen.

43. A kit or combination comprising the culture medium B according to claim 23 and/or the culture medium A according to claim 30.

44-50. (canceled)
